(19) United States
(12) Patent Application Publication
(10) Pub. No.: US 2025/0130174 A1
(43) Pub. Date: Apr. 24, 2025

(54) MACHINE LEARNING-ASSISTED DUAL-FUNCTION NANOPHOTONIC SENSOR FOR ORGANIC POLLUTANT DETECTION AND DEGRADATION

(71) Applicant: Trustees of Dartmouth College, Hanover, NH (US)

(72) Inventors: Junhu Zhou, West Lebanon, NH (US); Congran Jin, White River Junction, VT (US); Ziqian Wu, West Lebanon, NH (US); Xiaojing Zhang, Hanover, NH (US)

(21) Appl. No.: 18/924,364

(22) Filed: Oct. 23, 2024

Related U.S. Application Data
(60) Provisional application No. 63/545,359, filed on Oct. 23, 2023.

(51) Int. Cl. G01N 21/65; G01N 21/94; G01N 33/18

(52) U.S. Cl. CPC G01N 21/658; G01N 21/94; G01N 33/18; G01N 2201/1296

(57) ABSTRACT

A method including detecting contaminants in a water sample using a machine learning algorithm having a Laplacian operator configured to extract Raman peak data, a deep neural network, and a K nearest neighbors (KNN) cluster model. The method may include a test system having a silicon nanofiber film, a plurality of ZnO nanorods arranged in an array on the silicon nanofiber film, and a plurality of silver particles disposed on the plurality of ZnO nanorods. A water sample may be applied onto the test system, and the water sample may be measured using surface-enhanced Raman spectroscopy to generate the Raman peak data used in the machine learning algorithm.

### CROSS REFERENCE TO RELATED APPLICATIONS

This application claims priority to U.S. Provisional Application No. 63/545,359, filed on Oct. 23, 2023, the entire disclosure of which is incorporated herein by reference.

### BACKGROUND OF THE DISCLOSURE

Water is an essential resource for all living organisms, and access to clean water is crucial for maintaining good health. Data from the World Health Organization (WHO) reveals that 2 billion people lack access to safely managed water service as of 2020, including 282 million people with limited services and 122 million drinking surface water. Water pollution has become a serious threat to water quality due to economic development, global population increase, and climate changes. Organic pollutants, including pharmaceuticals, pesticides, organic dyes, detergents, and industrial waste, are of particular concern because they consume oxygen in water, generate toxic residues, and can persist in the water body for a long time due to their chemical stability.

Traditional water purification approaches encompass chemical precipitation, filtration, adsorption, ion exchange, chlorination, and distillation. Although these techniques are widely used in industrial settings, they have limitations such as lengthy processing times, low removal efficiencies, chemical resistance, and risks of re-contamination.

Monitoring water quality is crucial for environmental and human health on both large and small scales. However, detecting organic pollutants in water is challenging due to molecular variability and complexity, as well as the need for low limits of detection (LoD). Technologies such as photo-luminescence spectroscopy, high-resolution mass spectrometry (HRMS), high-performance liquid chromatography (HPLC), and surface-enhanced Raman spectroscopy (SERS) have been used to detecting water contaminants. SERS is extensively researched due to its label-free, ultrasensitive, fast, and versatile characteristics. Nevertheless, identifying complex Raman spectra presents challenges, including mixed features, complex datasets, instrument noise interference, and sample property effects. To extract meaningful information from the Raman spectrum data, various machine learning (ML) algorithms have been developed, including partial least square (PLS) regression, support vector machine (SVM), convolutional neural network (CNN), recurrent neural network (RNN), and deep learning (DL). DL is a type of ML algorithm that employs multiple hidden layers of neural networks to extract complex features from large and diverse datasets. These deep neural networks can efficiently learn and represent nonlinear relationships between input and output data without domain-specific knowledge, allowing accurate classification of Raman spectra for corresponding chemical compositions. However, detecting “out-of-distribution” (OOD) samples (i.e., class of unseen data points), which are different from the training data, remains a common challenge in DL. Despite this challenge, OOD detection is vital for ensuring the reliability of ML algorithms for SERS detection, particularly when training data is limited, and diverse chemicals may be present in the test samples. Current ML-assisted Raman detection techniques also struggle to discern mixtures of molecules and accurately characterize complex mixtures.

Thus, water purification and detection systems and methods with improved detection accuracy, purification efficiency, and cost efficiency are needed.

### SUMMARY OF THE DISCLOSURE

The present disclosure provides a dual-functional thin film, AgNP-ZnONR-SNF (Ag nanoparticle decorated, ZnO nanorod coated silica nanofibers) testing system. In an embodiment, the AgNP-ZnONR-SNF testing system may be used in water purification and organic pollutants sensing. The 3D fibrous structure of ZnONR-SNF provides a large surface area to volume ratio for piezo-catalytic and photo-catalytic degradation of organic pollutants under UV irradiation, which may achieve over 98% efficiency. In an embodiment, Ag nanoparticles decorated on ZnONR-SNF form “hotspots” that enhance the surface-enhanced Raman scattering (SERS) signal, which may result in an enhancement factor of 1,056 and an experimental detection limit of 1 pg/mL.

The present disclosure further provides a machine learning algorithm for the qualitative and quantitative detection of multiple contaminants that may achieve high accuracy (92.3%) and specificity (89.3%) without the need for preliminary processing of Raman spectra. In an embodiment, the machine learning algorithm may include a Laplacian operator, a deep neural network with two output modes to show the qualitative and quantitative detection results, and a K nearest neighbors (KNN) cluster model that is combined with the classification mode output to enable detection of novelty classes.

The present disclosure provides a water purification and detection material using a silica nanofiber (SNF) thin film decorated with ZnO nanorod (ZnONR) and silver nanoparticle (AgNP). An embodiment of the material system can efficiently degrade and remove organic pollutants while enabling rapid, quantitative, and label-free detection of these contaminants through SERS. A deep learning (DL) algorithm may be used to facilitate the qualitative, quantitative, and out-of-distribution (OOD) detection of mixed contaminants without the need of preliminary processing. A Laplacian operation may be implemented to effectively extract the Raman peak information from the original Raman spectra. The output of the neural network (NN) may be either a regression to show the concentration of multiple analytes or a classification to identify the analyte components and concentration levels, combined with K-nearest neighbor (KNN) for novelty class detection.

An embodiment of the present disclosure provides the use of zinc oxide (ZnO) in a water treatment. ZnO has photo- and piezo-catalytic properties and presents an ecofriendly material for processing contaminated water. An advantage of using ZnO in water treatment is its ability to react with persistent organic contaminants under natural sunlight, which contains UV light. Hence, it offers a more sustainable and eco-friendly approach for removing organic pollutants than traditional methods.

In an embodiment, ZnO may be fabricated into various nanostructures, such as nanospheres, nanorods, and nanoflowers. Further, in an embodiment, ZnO nanoparticles may be anchored on silica substrates which have robust mechanical strength and chemical stability. Growing ZnO on silica nanofibers (SNF) can increase the contact area, prevent nano pollution, and enable reusability.

Embodiments disclosed herein may be used for wastewater treatment.

Embodiments of the present disclosure include a method including detecting contaminants in a water sample using a machine learning algorithm. In an embodiment, the machine learning algorithm may include a Laplacian operator configured to extract Raman peak data, a deep neural network, and a K nearest neighbors (KNN) cluster model.

In embodiments of the present disclosure, the machine learning algorithm may perform the detecting using the Raman peak data.

In embodiments of the present disclosure. the deep neural network may include a first output mode and a second output mode to output detection results.

In embodiments of the present disclosure, the first output mode may be a classification mode to classify analyte components in the water sample and a concentration level of the water sample.

In embodiments of the present disclosure, the output may be a tensor with a 0 or a 1, wherein the tensor indicates the concentration of the water sample.

In embodiments of the present disclosure, the output of the first mode may be a five digit tensor, and the first four digits of the five digit tensor may correspond to a dye type.

In embodiments of the present disclosure, the second output mode may be a regression mode to detect a concentration of analytes present in the water sample.

In embodiments of the present disclosure, the second output may be a tensor with a number. In an embodiment, the tensor may indicate the concentration of the analytes present in the water sample.

In embodiments of the present disclosure, the KNN cluster model may be combined with the classification mode to classify the analyte components in the water sample.

In embodiments of the present disclosure, the method may further include measuring the water sample using surface-enhanced Raman spectroscopy to generate the Raman peak data.

In embodiments of the present disclosure, the contaminant may be an organic pollutant, an inorganic pollutant, a bacterial contaminant, or a virus.

In embodiments of the present disclosure, a non-transitory computer readable medium storing a program may be configured to instruct a processor to execute embodiments of the methods disclosed herein.

Further, embodiments of the present disclosure include a method including providing a test system, applying a water sample onto the test system, and detecting contaminants in the water sample using a machine learning algorithm. In embodiments, the test system may include a silicon nanofiber film, a plurality of ZnO nanorods arranged in an array on the silicon nanofiber film, and a plurality of silver particles disposed on the plurality of ZnO nanorods. In embodiments, the machine learning algorithm may include a Laplacian operator configured to extract Raman peak data, a deep neural network, and a K nearest neighbors (KNN) cluster model.

In embodiments of the present disclosure, the machine learning algorithm may perform the detecting using the Raman peak data.

In embodiments of the present disclosure, the deep neural network may include a first output mode and a second output mode to output detection results.

In embodiments of the present disclosure, the first output mode may be a classification mode to classify analyte components in the water sample and a concentration level of the water sample.

In embodiments of the present disclosure, the second output mode may be a regression mode to detect a concentration of analytes present in the water sample.

In embodiments of the present disclosure, the KNN cluster model may be combined with the classification mode to classify the analyte components in the water sample.

In embodiments of the present disclosure, the method may further include measuring the water sample on the test system using surface-enhanced Raman spectroscopy to generate the Raman peak data.

In embodiments of the present disclosure, the contaminant may be an organic pollutant, an inorganic pollutant, a bacterial contaminant, or a virus.

### BRIEF DESCRIPTION OF THE FIGURES

For a fuller understanding of the nature and objects of the disclosure, reference should be made to the following detailed description taken in conjunction with the accompanying figures.

FIG. 1A displays components of an embodiment of an AgNP-ZnONR-SNF system for organic pollutant sensing and degradation.

FIG. 1B displays a diagrammatic sketch of the photo- and piezo-catalytic degradation process.

FIG. 1C displays a schematic demonstration of an embodiment of the ML models disclosed herein.

FIG. 2A displays a schematic diagram of an embodiment of the present disclosure to showcase the synthesis procedure of the ZnONR and AgNP on electrospun SNF thin film.

FIG. 2B displays a scanning electron microscopy (SEM) image of the SNF.

FIGS. 2C and 2D display a scanning electron microscopy (SEM) image of the ZnONR-SNF.

FIG. 2E displays a scanning electron microscopy (SEM) image of AgNP on the top of the ZnONR.

FIG. 2F displays an energy dispersive spectrometry (EDS) mapping of an embodiment of the AgNP-ZnO-SNF with a SEM image.

FIG. 2G displays an energy dispersive spectrometry (EDS) mapping of a Zn element.

FIG. 2H displays an energy dispersive spectrometry (EDS) mapping of a O element.

FIG. 2I displays an energy dispersive spectrometry (EDS) mapping of an Ag element.

FIG. 2J displays the diameter distribution of embodiments of the SNF, AgNP, and ZnONR-SNF in a semi-logarithmic plot.

FIG. 2K displays an EDS spectrum of an embodiment of the AgNP-ZnO-SNF.

FIG. 2L displays the normalized UV-visible optical absorption spectrum of embodiments of ZnONR-SNF and AgNP-ZnO-SNF.

FIG. 3A displays photo-catalytic degradation results as described in Example 1.

FIG. 3B displays piezo-catalytic degradation results as described in Example 1.

FIG. 3C displays hybrid degradation results as described in Example 1.

FIG. 3D displays photo-catalytic and piezo-catalytic degradation kinetic curves of dye solutions catalyzed by an embodiment of the ZnONR-SNF, as described in Example 1.

FIG. 3E displays hybrid degradation kinetic curves of dye solutions catalyzed by an embodiment of ZnONR-SNF, as described in Example 1.

FIG. 3F displays degradation results of control groups without the ZnONR-SNF.

FIG. 3G displays a schematic illustration of the degradation process.

FIG. 3H displays a schematic graphic showing the piezo- and photo-catalytic degradation process of the ZnONR.

FIG. 3I displays a schematic diagram of the piezoelectric property of ZnO.

FIG. 4A displays the chemical structure of antibiotic Cip and dye MB.

FIG. 4B displays the Raman spectra of Cip solution of various concentrations.

FIG. 4C displays the Raman spectra of MB solution of various concentrations.

FIG. 4D displays the Raman intensity versus the concentration curve of Cip.

FIG. 4E displays the Raman intensity versus the concentration curve of MB.

FIG. 4F displays the SERS enhancement of MB dye on different substrates.

FIG. 4G displays the Raman signal mapping of the 1614 cm⁻¹ characteristic peak of the MB dye showing uniform intensity across an embodiment of the AgNP-ZnONR-SNF chip.

FIG. 4H displays the distribution of Raman intensity.

FIG. 5A displays the quantitative detection results of a three-component mixture from NN regression.

FIG. 5B displays qualitative detection results of NN regression.

FIG. 5C displays the statistical detection accuracy of each type of sample, as described in Example 1.

FIG. 6A displays the calculated minimum distance toward established clusters using KNN clustering method, as described herein.

FIG. 6B displays the calculated minimum distance toward established clusters using NN classifier and KNN clustering methods, as described herein.

FIG. 6C displays the accuracy of class points detection from KNN alone and NN classifier together with KNN.

FIG. 6D displays the statistics of class points detection using NN classifier and KNN.

FIG. 7A displays SEM images of a top view of ZnONR.

FIG. 7B displays SEM images of a side-view of ZnONR.

FIG. 7C displays AgNP on the top of the ZnONR.

FIG. 8A displays optical images of AgNP-ZnONR-SNF with 1000 magnification.

FIG. 8B displays 3D reconstruction of the AgNP-ZnONR-SNF thin film.

FIG. 9A displays the concentration-absorbance calibration curves of MB.

FIG. 9B displays the concentration-absorbance calibration curves of MO.

FIG. 9C displays the concentration-absorbance calibration curves of TB.

FIG. 9D displays the UV-Visible absorbance curves of MB under different UV irradiation time.

FIG. 9E displays the UV-Visible absorbance curves of MO under different UV irradiation time.

FIG. 9F displays the UV-Visible absorbance curves of TB under different UV irradiation time.

FIG. 9G displays the concentration-absorbance calibration curve of Cip.

FIG. 9H displays the photocatalytic degradation results of Cip under a UV lamp and sunlight.

FIG. 9I displays the normalized UV-Visible absorbance curves showing the characteristic peaks of different molecules.

FIG. 10 displays the results of a reusability test of the photocatalytic property of ZnONR-SNF thin film against MB, TB and MO.

FIG. 11A displays a simulation of the localized surface plasmon enabled electrical field enhancement of the Ag nanoparticle dimer of 40 nm diameter on the top surface of ZnONR with gap of 5 nm.

FIG. 11B displays a simulation of the localized surface plasmon enabled electrical field enhancement of the Ag nanoparticle dimer of 40 nm diameter on the top surface of ZnONR with gap of 10 nm.

FIG. 11C displays a simulation of the localized surface plasmon enabled electrical field enhancement of the Ag nanoparticle dimer of 40 nm diameter on the top surface of ZnONR with gap of 15 nm.

FIG. 11D displays an Ag nanoparticle dimer of 40 nm diameter on the side surface of ZnONR with gap of 5 nm.

FIG. 11E displays an Ag nanoparticle dimer of 40 nm diameter on the side surface of ZnONR with gap of 10 nm.

FIG. 11F displays an Ag nanoparticle dimer of 40 nm diameter on the side surface of ZnONR with gap of 15 nm.

FIG. 11G displays an Ag nanoparticle dimer of 40 nm diameter at the edge of ZnONR with gap of 5 nm.

FIG. 11H displays an Ag nanoparticle dimer of 40 nm diameter at the edge of ZnONR with gap of 10 nm.

FIG. 11I displays an Ag nanoparticle dimer of 40 nm diameter at the edge of ZnONR with gap of 15 nm.

FIGS. 12A-12F displays a simulation of the localized surface plasmon enabled electrical field enhancement of the Ag nanoparticle with different diameter and pair number under 532 nm light.

FIG. 13A displays results of the extinction cross-section area of the AgNP number from 0 to 3 (40 nm diameter, 5 nm gap, on the top surface).

FIG. 13B displays the extinction cross-section area of the AgNP with different diameter (dimer, 5 nm gap, on the top surface).

FIG. 13C displays the extinction cross-section area of the AgNP with different gap (dimer, 40 nm diameter, on the top surface).

FIG. 13D displays the extinction cross-section area of the AgNP at different locations (dimer, 40 nm diameter, 5 nm gap).

FIG. 14A displays SERS spectra of Cip, MO, TB and MB.

FIG. 14B displays SERS spectra of the combinations of MB, TB and Cip.

FIG. 15A displays the qualitative detection results of MB & TB mixtures, where the colors of points indicate their true classes and the positions of points (the quadrant of their coordinates) refer to the predictive classes.

FIG. 15B displays the statistical results of prediction compared with ground truth.

FIG. 16 displays the hierarchical cluster analysis (HCA) of Raman spectra from 15 groups of mixtures containing Cip, TB and MB.

FIG. 17 displays the ablation test to validate the rationality of central difference calculation prior treatment.

FIGS. 18A-18B displays the confusion matrix to illustrate the training and testing classification accuracy obtained from the NN regressor, both of which were computed using 100 example samples.

FIGS. 19A-19B displays the confusion matrix to illustrate the classification accuracy of the NN classifier, showcasing both the training and testing performance.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, process step, and electronic changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

The steps of the method described in the various embodiments and examples disclosed herein are sufficient to carry out the methods of the present invention. Thus, in an embodiment, the method consists essentially of a combination of the steps of the methods disclosed herein. In another embodiment, the method consists of such steps.

The present disclosure further provides a secure, high-throughput water purification-monitoring test system and methods that can quickly, accurately, quantitatively, and automatically detect multiple water contaminants (e.g., organic dyes, antibiotics) using a single, rapid test system. The water samples can be easily collected from the wastewater and no sample pretreatment is required. In an embodiment, surface enhanced plasmonic sensing using Raman spectroscopy, or SERS, may be used. In an embodiment of the present disclosure, the main module of the degradation-sensing platform includes a silver (Ag) or gold (Au) nanoparticle decorated ZnO nanorod coated silica nanofiber matrix (Ag/AuNP-ZnONR-SNF). The Ag nanoparticles are used for their strong antibacterial and plasmonic sensing amplification properties. ZnO has strong degradation properties and may be used to degrade chemically complex organic pollutants. Further, the photo-catalytic, piezo-catalytic, and antibacterial properties of ZnO make it a powerful material in processing contaminated water. Silica nanofibers provide a 3D matrix to increase the surface-to-volume ratio and increase the water purification efficiency.

In an embodiment, a machine learning (ML) algorithm may be incorporated to achieve the automatic, quantitative analysis of multiplex detection of the contaminant(s) without the need for trained staff. The machine learning algorithm may be used to analyze measurements from the surface-enhanced Raman spectroscopy. Further, the machine learning algorithm may be a prior embedded neural network, but other machine learning algorithms may be used. Embodiments may include a non-transitory computer readable medium that may store a program to instruct a processor to execute the machine learning algorithm disclosed herein.

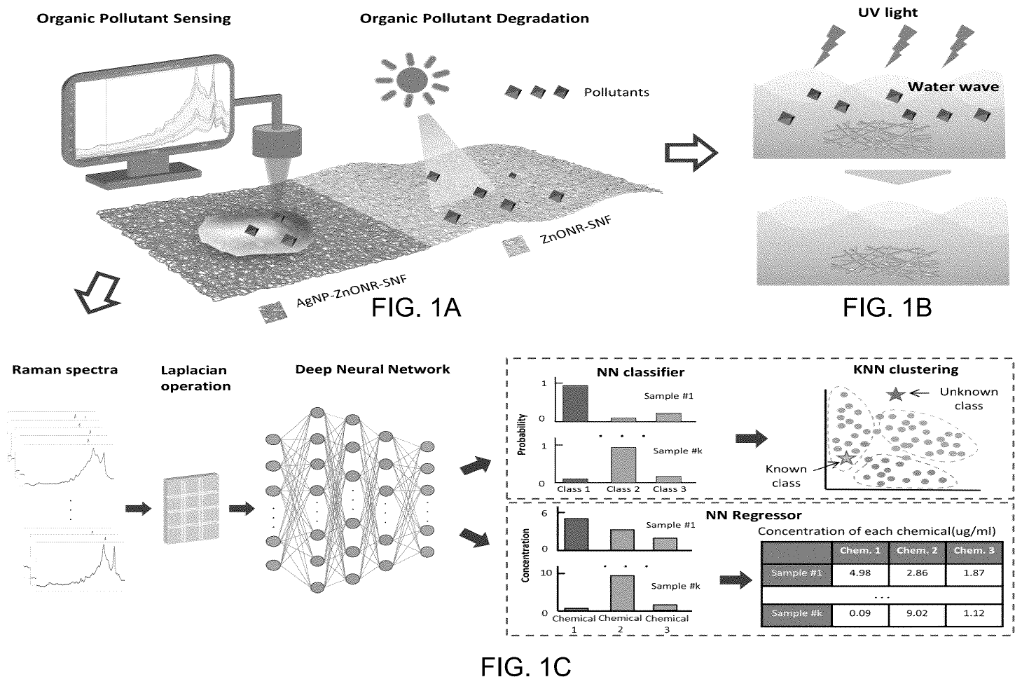

FIGS. 1A-1C display an embodiment of the present invention. Specifically, FIGS. 1A-1C display the schematic diagram of the pollutants degradation sensing and machine learning (ML) algorithm assisted data interpretation process of the dual functional device. FIG. 1A displays the components of the AgNP-ZnONR-SNF system. Embodiments of the present disclosure may be used for organic pollutant sensing and degradation. FIG. 1B displays an embodiment of the photo-catalytic and piezo-catalytic degradation process. FIG. 1C displays a schematic demonstration of an embodiment of the machine learning models disclosed herein.

Embodiments of the present disclosure allows for an improvement of the current water contaminant degradation and monitoring technology by providing an innovative degradation-sensing technique. The embodiments disclosed herein provide means to address the challenges in water purification and monitoring in the current global water pollution crisis.

The photo-catalytic, piezo-catalytic, and antibacterial properties of ZnO makes it an eco-friendly material in processing contaminated water. One advantage of ZnO as a water treatment material is that natural sunlight containing UV light can induce its photo-catalytic reactions with persistent organic contaminants. Because of this, ZnO is a greener and more eco-friendly approach for removing organic pollutants than other techniques. However, these characteristics of ZnO in processing wastewater cannot be fully utilized without a designed material and engineered system. ZnO has been fabricated into various nanostructures such as nanospheres, nanorods, and nanoflowers. Despite these efforts, improvements are possible, such as with degradation efficiency, prevention of secondary contamination, and repeatability. ZnO nanoparticles can suffer from low surface-to-volume ratio, possible secondary contamination, and difficulty in repeated use. Embodiments of the present disclosure overcome these issues.

In addition to water purification, water quality monitoring is equally important to the environment and human health both at large and small scale. Organic pollutant detection can be challenging because of the variability and complexity of the molecules and the low limit of detection (LoD). Technologies including photo-luminescence spectroscopy, high-resolution mass spectrometry (HRMS), high-performance liquid chromatography (HPLC), and surface-enhanced Raman spectroscopy (SERS) have been used for the detection of water pollutants. Among them, SERS is widely used because of its label-free, ultrasensitive, fast, and feasible characteristics. However, mixed features, complex datasets, interferences from instrumentation noise, and sample properties can make the identification of complex Raman spectrum a challenging tool. To extract meaningful information from the Raman data, various machine learning (ML) algorithms have been developed for Raman spectrum identification, including partial least square (PLS) regression, support vector machine (SVM), convolutional neural network (CNN), recurrent neural network (RNN), prior embedded neural network (embedded neural network, neural network, or NN), and Deep learning (DL). Deep learning is a versatile method to be applied in various biomedical scenarios. Because of the multiple hidden layers of neural network, DL method is efficient in extracting complex features using multi-layer structure even without the expertise from chemists or spectroscopists.

In the embodiments the test system can accurately, quantitatively, and rapidly detect multiple contaminants at the same time using machine learning methods disclosed herein.

Surface plasmonic resonance can be used for quantitative multiplex water contaminant sensing. Embodiments of the sensor are based on surface plasmonic resonance (SPR) for quantitatively detecting contaminating substances in water by identifying their molecular bonding using Raman spectroscopy. Compared to a conventional water quality monitor, this technique is more accurate, faster (in minutes), more cost-effective, and requires less expertise to operate, as compared to existing methods.

In an embodiment, the test system may perform the test on 50 samples in only a few seconds. SERS detection using the embodiments disclosed herein can be completed within one minute.

Embodiments of the nanomaterial-based platform can be constructed for optimal sensing performance. Conventional surface enhanced plasmonic sensing platforms have metal nanoparticles randomly distributed on a 2D surface such as a silicon wafer. Sensors based on this material structure have limitations in signal strength, sensitivity, and LoD. Embodiments disclosed herein include a three-layer, 3D nanostructure in which Ag or Au nanoparticles are decorated on ZnO nanorods that were grown on a silicon nanofiber. This 3D structure increases the plasmonic signal strength and, as a result, improves the sensitivity and LoD.

Embodiments of the disclosure may be used for quantitative, label-free, and rapid detection of the contaminants using SERS. The 3-dimensional (3D) matrix of the ZnO nanorod coated silica nanofiber and the silver nanoparticles (AgNPs) decorated on the ZnO nanorod may enhance the Raman signal of the sensing contaminants by the large amount of “hotspots” between adjacent AgNPs that maximize plasmonic coupling.

A principle behind the degradation ability of organic pollutants is based on the advanced oxidation processes (AOPs) generated by the photo-catalytic and piezo-catalytic properties of ZnO nanorods. AOPs are a set of chemical procedures including in-situ generation of highly reactive and oxidizing radical species which can destroy the organic pollutants.

In an instance, a mechanism of photo-catalytic degradation includes diffusing organic pollutants from the liquid phase to be absorbed to the surface of ZnONR. Then ZnONR is irradiated by the UV light with energy larger than its bandgap energy, which promotes electrons (e⁻) from valence band (VB) to conduction band (CB) and leaves holes (h⁺) in the VB. Next, the photogenerated electron-hole (e⁻/h⁺) pairs can migrate to the ZnO nanorod surface, reacting with water and hydroxide ions (by h⁺) and oxygen (by e⁻) to generate reactive oxygen species (ROS) including hydroxyl radical (·OH) and superoxide anion (O₂^{·-}). Finally, the ROS can directly oxidize organic pollutant molecules.

In an instance, in a mechanism of piezo-catalytic degradation, upon interaction with mechanical force (generated by the water flow), ZnONR can be deformed and a strain field may be created by the deflection with the outer side being stretched while the inner side is compressed. An electric field along the ZnONR is then created inside through the piezoelectric effect producing surface charge accumulation at the opposite surface. It allows (e⁻/h⁺) to migrate to the ZnONR surface and triggers subsequent reactions similar to the photocatalytic degradation process. The piezoelectric potential also contributes to the adsorption of charged organic molecules.

Embodiments of the present disclosure further includes a machine learning algorithm to provide quick, quantitative, and accurate results without the need for trained professionals. The resulting SERS signals are plotted in a spectrum. Characteristic signal peaks are used to identify the molecules of the substances in the sample. However, mixed features, complex datasets, interferences from other substances in the sample, and instrumentation noise make data interpretation challenging, even for experienced technicians.

In an embodiment, Raman spectra obtained by the test system may contain intrinsic vibrational fingerprints that can aid in analyte identification. To extract meaningful information (i.e., water contaminants) quickly and automatically from the Raman data, a machine learning algorithm can enable exploration of complex characteristics from large raw datasets and can achieve accurate results.

The SERS enhancement of the sensor can occur from 1050 cm⁻¹ to 1650 cm⁻¹. This is an enhancement over other ranges. This can potentially be overcome by changing the nanostructure of the sensor. For instance, re-designing the ZnO nanostructure, changing the size of silver nanoparticles, and/or adding gold nanoparticles can improve enhancement.

In an embodiment of the machine learning algorithm, the algorithm may include a Laplacian operator, a deep neural network with two output modes to show the qualitative and quantitative detection results (which may refer to a neural network regression and neural network classification detection results), and a K nearest neighbors (KNN) cluster model that is combined with the classification mode output to enable detection of novelty classes.

In an embodiment, the Laplacian operator may be a second-order differential operator that is used for analyzing spatial structures. The Laplacian operator may be directly applied to Raman spectra data, as it inherently emphasizes regions of rapid intensity changes, such as peaks, which makes it well-suited for enhancing signal features without requiring complicated preliminary data processing. The Laplacian operator may be applied to Raman spectra having any number of points.

In an embodiment, the deep neural network may be a fully-connected deep neural network. After the Laplacian operation, the treated data may be taken into the deep neural networks to output the detection results through one or more modes, such as two modes. In the first mode, the NN may be a classification (fully connected layers with sigmoid as activation layer combined with RNN) to show the analyte components and concentration level. The output of NN may be a tensor with part of the tensor showing the possibility that the sample belongs to a specific class and part of the tensor either 0 or 1 to indicate if the specific chemical is above a typical cutoff concentration. In the second mode, the NN may be a regression (fully connected with rectified linear unit (ReLU) as activation layer combined with RNN) to directly show the concentration of multiple chemicals that were included in the sample. The output may be a tensor with arbitrary numbers showing the concentration of each chemical, where a small number may indicate that the specific analyte does not exist. Both the two modes may output the qualitative and quantitative detection of mixed analytes to satisfy different application requests. In embodiments, the model may be implemented with PYTHON.

In an embodiment, the KNN cluster model may be used for classification tasks by finding the K closest neighbors and making decisions based on their labels. To allow for out-of-distribution detection and referring to the detection of novelty class, a KNN clustering method may be incorporated with the NN classifier. Clusters that may be in accordance with the known classes from the NN classifier output may be established through the KNN clustering model. By calculating the distance of a test point to the established clusters after clustering, the test point may be correctly identified as seen class data or unseen class data.

ZnO can be used as a platform synthesis material due to its biosafety, versatility, and low-cost manufacturing. ZnO has optoelectronic and electrochemical properties that can enable various sensing and engineering applications. ZnO arrays can provide a platform for SERS-based sensing of various substances because ZnO offers a higher reproducibility and stability, larger surface-to-volume ratio, and a 3D platform.

Embodiments of the present disclosure include a test system. In an embodiment of the sensor, one or more ZnO nanorods can be grown on a silicon nanofiber film (SNF) using a hydrothermal technique. Silver particles can be decorated on the ZnO nanorods using ultraviolet (UV) irradiation. The size of the ZnO nanorods and the silver particles can be controlled during the process. Simulations using a finite element method (FEM) can be carried out to confirm the plasmonic effect of the fabricated structure. A water sample may be applied to the test system, and a concentration of a pollutant in the water sample may be determined using surface-enhanced Raman spectroscopy of the test system.

A two-step hydrothermal growth and UV irradiation protocol can be used to synthesize ZnO arrays decorated with silver particles or other particles on a silicon nanofiber film (or silicon chip). These particles contribute to the local surface plasmonic effect and an increase in silver SERS enhancement “hotspots.”

The SNF size can vary. In an embodiment, the SNF is 12 cm in diameter, but other shapes and sizes are possible. The SNF may be sized such that it can be easily handled by laboratory staff. The thickness of the SNF may be controlled by the electrospinning time of the film. In an embodiment, the SNF may be from 0.5 mm to 1 mm thick. Other electrospun nanofibers also may be used as the substrate.

The ZnO nanorods may have a homogenous dispersity. In an embodiment, the ZnO nanorods can have dimensions in the nm to μm range. The average width of the cross section of the ZnO nanorod may be from 200 nm to 3 μm, including all 0.1 nm values and ranges therebetween (e.g., 2±0.3 μm). If the cross-section of the ZnO nanorod is larger than 3 μm, then there may be more particles on the top surface of the ZnO nanorod than on the side surfaces, which would weaken the strength of a sensing signal. If the cross-section of the ZnO nanorod is smaller than 200 nm, then the ZnO nanorod may not be capable of supporting enough particles, which also would weaken the strength of a sensing signal.

In an embodiment, the height of the ZnO nanorods may be from 1 to 5 μm, including all nm values and ranges therebetween (e.g., approximately 2 μm). Without intending to be bound by any particular theory, the height of the ZnO nanorods may be on the micron scale because such a size is suitable for local signal enhancement, but large enough to support a desirable/sufficient number of particles. In various examples, the ZnO nanorods have a height to width ratio greater than 1. For example the height to width ratio may be >1:1 to 5:1, including all 0.1 ratio values and ranges therebetween. A larger height to width ratio would mean the ZnO nanorod is too narrow to support enough particles, which would weaken the strength of a sensing signal. A smaller height to width ratio would result in more particles on the top surface of the ZnO nanorods than on the side surfaces, which would weaken the strength of a sensing signal.

The cross-section of the ZnO nanorods can be different shapes. For example, the ZnO nanorods may have a hexagonal cross section, but other circular, ovoid, or polygonal shapes are possible.

Without intending to be bound by any particular theory, it is considered that the ZnO nanorods operate as scaffold. Silver and/or gold particles may be disposed (e.g., deposited) such that these particles cover (e.g., coat) at least a portion of a surface of the pillar or the whole pillar surface. The ZnO nanorods can be vertically arranged.

In an embodiment, the ZnO nanorods may be arrayed on a substrate (e.g., the thin film) as a result from the hydrothermal ZnO growth process. The density of the array of ZnO nanorods may be controlled by controlling the seeding density (the concentration of the seeding solution and the spin-coating speed of the seeding process) in the process. In various embodiments, there are about 10-12 pillars (e.g., 10, 11, or 12) in every 100 μm² area, though higher or lower densities are possible depending on the size of the pillar. Without intending to be bound by any particular theory, it is considered that the higher the density the more enhancement on the signal, and, thus, the better sensitivity and lower detection of limit of the chip.

While ZnO nanorods are used, other materials also can be used in the pillars or as a scaffold for the particles, such as titanium dioxide (TiO₂).

In embodiments of the present disclosure, silver and/or gold nanoparticles are disposed on the ZnO nanorods. The ZnO nanorods can serve as a scaffold for the silver particles. In an embodiment, the particles are disposed on the top surface of the ZnO nanorods, but the particles also can be disposed on the side surface of the ZnO nanorods. The side surface can be between the top surface and the substrate (e.g., silicon nanofiber film). Attaching particles on the side surface and the top surface can increase sensitivity.

The silver or gold particles may have various shapes. In an embodiment, the particles may be the same shape or may be different shapes. For example, the particles may be spherical, ellipsoidal, or spike-shaped. Spherical particles may be easier to fabricate and can provide sufficient results. While other shapes (e.g., ellipsoid, spike-shape) may give stronger signal due to their aspect ratio and spike features, these particles are usually more difficult to fabricate.

In an embodiment, the silver or gold particles may be disposed in similar densities or different densities on the top side and side of the nanorods. Without intending to be bound by any particular theory, it is considered that the higher the density of particles, the better the local enhancement of the signal until the particles are too close to touch each other. For example, “hot spots” may occur when the particles are within 100 nm of each other. However, local enhancement may occur on single (or separate) particles. Hot spots or local enhancement effect will increase the signal intensity and, thus, improve the sensitivity of the chip as well as lower the limit of detection of the chip.

The particles may have various sizes. In an embodiment, the particles may be roughly the same size (e.g., a homogenous size dispersity) or different sizes. The particles may have an average diameter of 20 to 100 nm, including all 0.1 nm values and ranges therebetween (e.g., 50±8 nm). Without intending to be bound by any particular theory, it is considered the smaller the particles, the more pronounced the local enhancement. However, when the particles get too small, they cover a smaller area and decrease the enhancement effect.

While silver particles are disclosed, gold particles, platinum particles, or other particles that include precious metals can be used. A mixture of particles that each include a precious metal can be used in an embodiment. For example, the mixture can include silver and gold, silver and platinum, or gold and platinum. The mixture also can include silver, gold, and platinum. These mixtures optionally can include other precious metals or other metals.

Parts of the embodiments of the present disclosure may include, be run with, or be operated by one or more processors that may include any processor or processing element known in the art. For the purposes of the present disclosure, the term “processor” or “processing element” may be broadly defined to encompass any device having one or more processing or logic elements (e.g., one or more micro-processor devices, one or more application specific integrated circuit (ASIC) devices, one or more field programmable gate arrays (FPGAs), or one or more digital signal processors (DSPs)). In this sense, the one or more processors may include any device configured to execute algorithms and/or instructions (e.g., program instructions stored in memory). In one embodiment, the one or more processors may be embodied as a desktop computer, mainframe computer system, workstation, image computer, parallel processor, networked computer, or any other computer system configured to execute a program configured to operate or operate in conjunction with the test system, as described throughout the present disclosure. Therefore, the above description should not be interpreted as a limitation on the embodiments of the present disclosure but merely as an illustration. Further, the methods described throughout the present disclosure may be carried out by a single processor or, alternatively, multiple processors. Additionally, the processors can be housed in a common housing or within multiple housings.

The present disclosure may also include a memory medium. The memory medium may include any storage medium known in the art suitable for storing program instructions executable by the associated one or more processors. For example, the memory medium may include a non-transitory memory medium. By way of another example, the memory medium may include, but is not limited to, a read-only memory (ROM), a random-access memory (RAM), a magnetic or optical memory device (e.g., disk), a magnetic tape, a solid-state drive and the like. It is further noted that memory medium may be housed in a common controller housing with the one or more processors. In one embodiment, the memory medium may be located remotely with respect to the physical location of the one or more processors and controller. For instance, the one or more processors of a controller may access a remote memory (e.g., server), accessible through a network (e.g., internet, intranet and the like).

Examples are included with this description. The examples are not meant to be limiting. While organic pollutants are disclosed, the embodiments disclosed herein can be used for inorganic pollutants, bacterial contaminants, or viruses.

Although the present disclosure has been described with respect to one or more particular embodiments, it will be understood that other embodiments of the present disclosure may be made without departing from the scope of the present disclosure.

The following examples are presented to illustrate the present disclosure. They are not intended to be limiting in any matter.

### Example 1

This example provides a description of embodiments of a dual-functional AgNP-ZnONR-SNF nanophotonic sensor that combines electrospinning, hydrothermal, and wet-chemical synthesis techniques.

Embodiments of the ZnONR-SNF thin films have shown high efficiency in degrading organic dyes (MB, TB, and MO) under low-powered UV and mechanical irradiation, while also being reusable. The results of the following example demonstrate the potential of this low-cost and eco-friendly system and method for water decontamination against organic pollutants. Additionally, the vertically aligned ZnONR provide suitable geometry to deposit Ag nanoparticles, which generates high-density hot spots for SERS enhancement, enabling the detection of nano-molar concentrations of both organic dyes (MB) and antibiotics (Cip) using AgNP-ZnONR-SNF thin films as a SERS substrate. In an embodiment, an embedded machine learning algorithm may be used for both qualitative and quantitative pollutants detection based on the Raman spectra obtained from AgNP-ZnONR-SNF thin films. In this example, the machine learning algorithm had an accuracy of 92.3% in qualitative detection and 90.8% in quantitative classification. This label-free dual-functional nanophotonic sensor, assisted by a machine learning algorithm, may be sensitive, fast, and portable, holding promise for environmental monitoring and sustainability studies.

### Materials and Methods

This example used the following chemicals and reagents: Tetraethyl orthosilicate (TEOS, 98%), formic acid, ethanol, Polyvinylpyrrolidone (PVP, M_{w}=1,300,000 g mol⁻¹), Zinc acetate ((CH₃CO₂)₂Zn, 99.99%), zinc nitrate hexahydrate (Zn(NO₃)₂·6H₂O, 98.0%), hexamethylenetetramine (C₆H₁₂N₄, HMTA, ≥99.0%), silver nitrate powder (AgNO₃, ≥99.0%), Methylene blue (MB, C₁₆H₁₈ClN₃S·xH₂O, M_{w}=319.85 g mol⁻¹), Trypan Blue (TB, C₃₄H₂₄N₆O₁₄S₄Na₄, M_{w}=960.81 g mol⁻¹), Methyl Orange (MO, C₁₄H₁₄N₃NaO₃S, M_{w}=327.33 g mol⁻¹) and Ciprofloxacin (Cip, C₁₇H₁₈FN₃O₃, M_{w}=331.34 g mol⁻¹) were purchased from Sigma-Aldrich Inc. Deionized (DI) water was from a Milli-Q water ultrapure water purification system.

### Fabrication of Silica Nanofiber

The silica nanofiber thin film was prepared through electrospinning according to a reported protocol with modifications. In a typical run, 1.9 g of TEOS, 3.15 g of ethanol, 2.0 g of water and 0.04 g of formic acid were mixed with 0.9 g of PVP. The mixture was stirred for one hour at room temperature until a transparent solution was formed. The solution was electrospun at a feed rate of 0.9 mL/h through a 22 G stainless needle under a high voltage (16 kV). The silica nanofibers were collected by a flat aluminum plate collector 10 cm away from the needle tip. The silica nanofiber thin film was peeled off from the aluminum foil gently and was then subjected to the calcination for three hours at 800° C. to remove PVP and other solvent residues.

### ZnO Nanorods Growth

The ZnO nanorods were created based on a seeding-growth method. First, the silica nanofiber thin film was immersed in 5 mM zinc acetate solution (in DI water) and then vacuumized to remove air bubbles. It was then transferred into an oven at 180° C. for 20 min for thermal decomposition of the zinc acetate to create ZnO seeds. This process was repeated 3 times. Then, zinc nitrate hexahydrate (35 mM) and HMTA (25 mM) were added to 45 mL of DI water to provide the hydrothermal growth solution. Next, the seeded thin film was placed in the growth solution in a beaker which was covered by aluminum foil and placed in a water bath for three hours at a temperature of 90° C. This growth cycle was repeated twice to form the ZnO nanorods. Finally, the thin film was rinsed with DI water to remove excessive ZnO residuals and dried in an oven at 50° C.

### Silver Nanoparticle Decoration

Ag nanoparticles were fabricated on top of the ZnO nanoarray by UV irradiation. The fabricated thin film was immersed in a 5 mM AgNO₃ solution (in DI water) and irradiated under a UV lamp (365 nm, 30 W) for 30 minutes. Then the film was washed with DI water and dried in an oven at 50° C.

### SEM and EDS Characterization

The morphologies of SNFs, ZnONR-SNF and AgNP@ZnONR-SNF were studied by scanning electron microscopy (SEM) performing on a FEI Helios 5CX dual beam scanning electron microscope operating at 5 kV. The energy-dispersive X-ray spectroscopic (EDS) ejected measurements and the chemical mapping were performed with the SDD X-ray detector (Oxford®) attached to the SEM microscope operating at 10 kV.

### Photo-Catalytic and Piezo-Catalytic Degradation

All organic molecules were dissolved in distilled water at a concentration of 10 μg/mL under sonication at room temperature. Calibration curves were obtained by considering the characteristic UV-Vis absorbance values (MB at 664 nm, TB at 590 nm, MO at 463 nm, 2,4-D at 282 nm, and Cip at 270 nm), obtained from a series of diluted solutions at prefixed concentration values (SI Figure). ZnONR-SNF films were cut into 0.8×0.8 cm² pieces and employed in glass vials with 3 mL of pollutant solutions for each degradation experiment. The control experiments were carried out in the absence of ZnONR-SNF films. Photocatalytic degradation experiments were carried out by applying a 30 W UV lamp (irradiated from upper side of the vials) at a fixed distance of around 5 cm. In piezo-catalytic degradation experiments, vials were fixed on the MTS 2/4 digital shaker orbiting at 300 rpm. In hybrid experiments, vials were fixed at the shaker and irradiated by the UV lamp simultaneously. All experiments were performed in the dark room at room temperature.

The degradation efficiency was measured by means of light absorbance. First, a 30 μL solution was withdrawn from the vials at a two hour time interval (0 hours, 2 hours, 4 hours, 6 hours, 8 hours) and placed in a 96 well UV-Star microplate. Then the light absorbance was read by a microplate reader (TECAN SPARK 10M) at each characteristic peak wavelength. According to the light absorbance readout and recorded calibration curve obtained previously, the concentration of the organic pollutants after degradation was finalized. The degradation efficiency was calculated by the following equation:

$$Degradation efficiency = \frac{{C}_{0} - C}{{C}_{0}} \times 100 \%$$

where C₀ was the initial concentration and C was the measured concentration at different times.

### SERS Detection

The SERS measurements were processed in a liquid system. The AgNP@ZnONR-SNF was first cut into a square piece (0.5 cm×0.5 cm). Then the substrate was focused with a 10 times objective lens. Next, the sample aqueous solution (10 μl) was added into the piece. Finally, SERS signals were obtained point-by-point from the 15×15 grid using a 532 nm laser as an excited source.

### Machine Learning Algorithm Development

In this example, the machine learning algorithm included a Laplacian operator, a deep neural network with two output modes to show the qualitative and quantitative detection results (which may refer to the neural network regression and neural network classification detection results, as shown in Table 1), and a K nearest neighbors (KNN) cluster model that is combined with the classification mode output to enable detection of novelty classes.

In this example, each collected Raman spectrum contained 238 points in the Raman shift axis to show the entire Raman shifting information of chemicals in the samples. The Laplacian operator was directly applied to the Raman spectra, as it inherently emphasized regions of rapid intensity changes, such as peaks, making it well-suited for enhancing signal features without requiring complicated preliminary data processing.

After the Laplacian operation, the treated data were taken into the deep neural networks to output the detection results through the two modes. In the first mode, the NN was a classification (fully connected layers with sigmoid as activation layer combined with RNN) to show the analyte components and concentration level. The output of NN was a tensor with part of the tensor showing the possibility that the sample belongs to a specific class and part of the tensor either 0 or 1 to indicate if the specific chemical was above a typical cutoff concentration. In the second mode, the NN was a regression (fully connected with rectified linear unit (ReLU) as activation layer combined with RNN) to directly show the concentration of multiple chemicals that were included in the sample. The output was a tensor with arbitrary numbers showing the concentration of each chemical, where a small number indicated that the specific analyte does not exist. Both the two modes may output the qualitative and quantitative detection of mixed analytes to satisfy different application requests. In embodiments, the model may be implemented with PYTHON.

To allow for out-of-distribution detection and referring to the detection of novelty class, a KNN clustering method may be incorporated with the NN classifier. Clusters that were in accordance with the known classes from the NN classifier output were established through the KNN clustering model. By calculating the distance of a test point to the established clusters after clustering, the test point was correctly identified as seen class data or unseen class data.

This example utilized PyTorch, along with its associated toolsets including Scikit-learn, NumPy, and Skorch, to implement the disclosed machine learning models. The disclosed model was executed on a standard workstation equipped with an Intel® Core® i9-9980 XE CPU running at 3.00 GHz, featuring 18 CPU cores, and an 8 GB NVIDIA GeForce RTX 2080Ti.

During each training process, the training and testing data were randomly divided. To ensure reliable outcomes, this process was repeated ten times, calculating the final error and prediction accuracy across these ten training iterations. For instance, in the experiments involving the regressor model, a 10-fold cross-validation approach was used using a total of 1095 data samples. This dataset was further divided into 995 training samples and 100 testing samples. Additional details regarding the sizes of the training and testing datasets are shown in Table 1.

TABLE 1
Parameters of neural networks and training data.

| | NN classification | NN regression |
|---|---|---|
| Prior treatment | Laplacian operation | |
| Fully connected layer structure | 128, 64, 32, 16 | |
| RNN layer number | 2 | |
| Training sample size | 400 | 995 |
| Test sample size | 65 | 100 |
| Activation function | Sigmoid | ReLU |
| Loss criterion | Cross-entropy | L1Loss |
| Optimizer | Adam | |
| Learning rate | 0.01 | 0.001 |
| Training epochs | 1000 | 2000 |
| Qualitative accuracy | 92.3% | 92% |
| Quantitative accuracy | 90.8% | MSE = 0.327 |

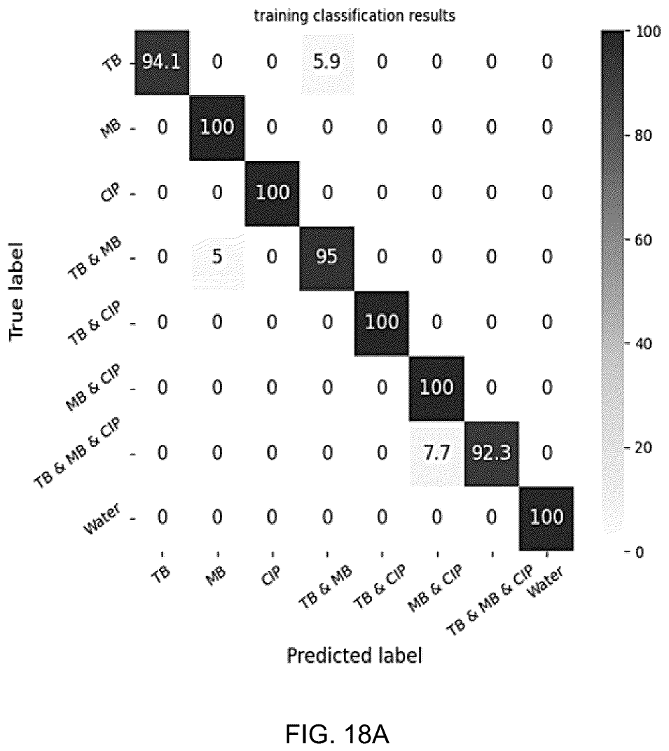

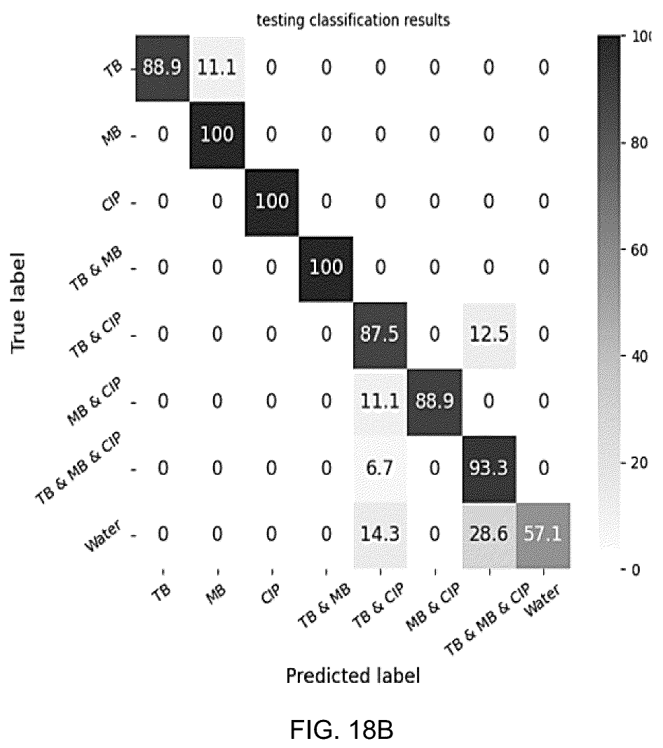

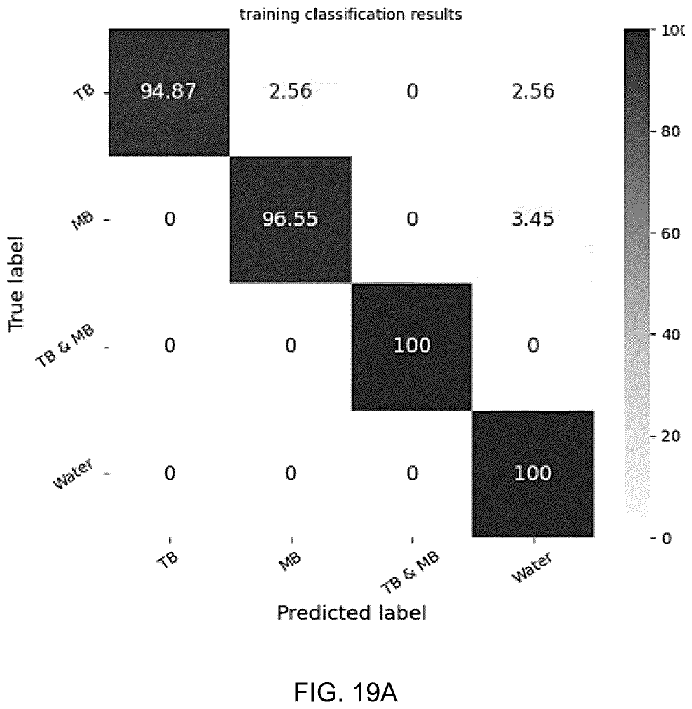

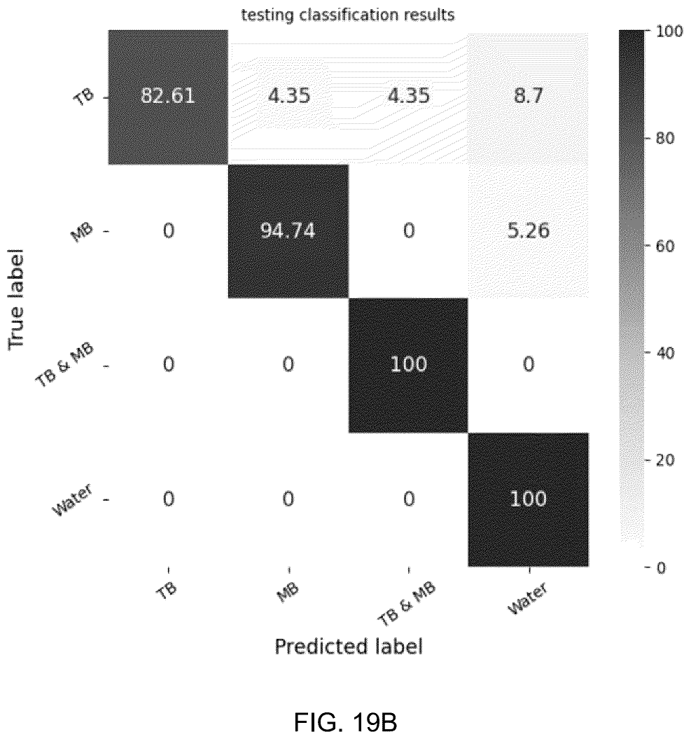

Furthermore, FIGS. 18A, 18B, 19A, and 19B present the confusion matrices pertaining to both the training and testing samples. Specifically, in FIGS. 18A and 18B, the confusion matrix illustrates the training and testing classification accuracy obtained from the NN regressor, both of which were determined on a computer using 100 example samples. In FIGS. 19A and 19B, the confusion matrix illustrates the classification accuracy of the NN classifier, showcasing both the training and testing performance. The training accuracy was computed using 100 samples, while the testing accuracy was assessed using 65 samples.

### Results and Discussion

### Characterization

The electrospun SNF thin film was collected from aluminum foil and had a diameter of approximately 12 cm. Specifically, the electrospun SNF was formed on aluminum foil and the SNF thin film was peeled from the aluminum foil. In an embodiment, the SNF thin film may be flexible. The scanning electron microscopy (SEM) was performed on a FEI Helios 5CX dual beam scanning electron microscope operating at 5 kV. The energy-dispersive X-ray spectroscopic (EDS) measurements and the chemical mapping were performed with the SDD X-ray detector (Ametek®) attached to the TESCAN Vega3 scanning electron microscope operating at 30 kV.

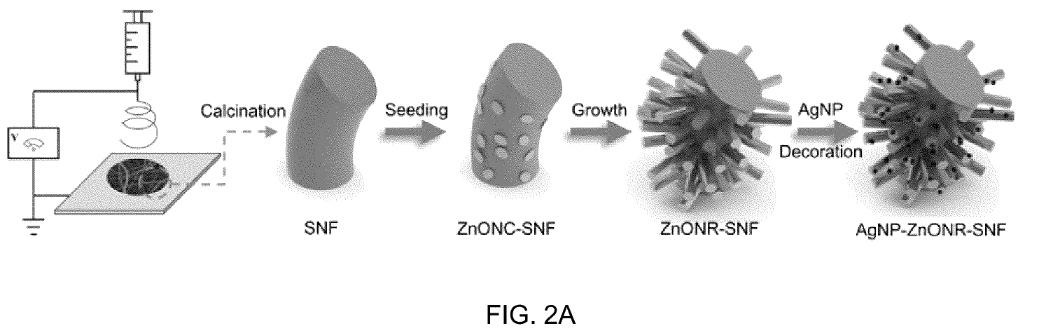

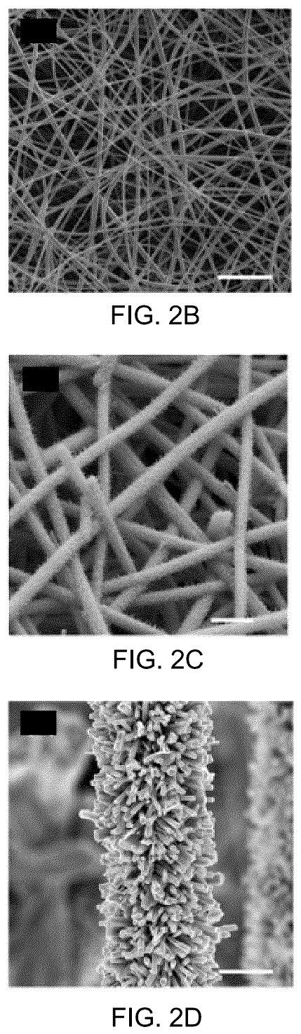

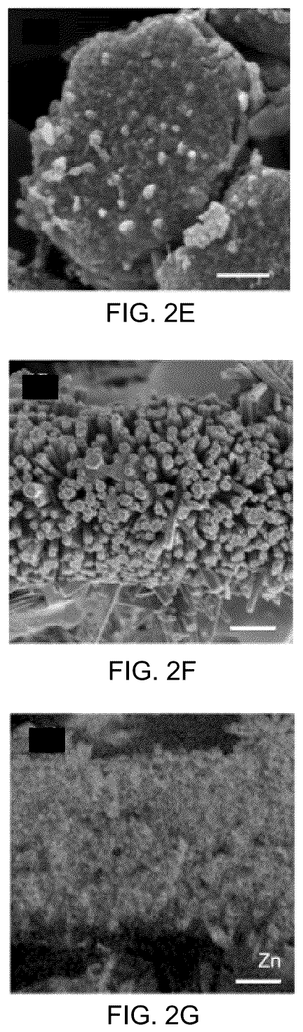

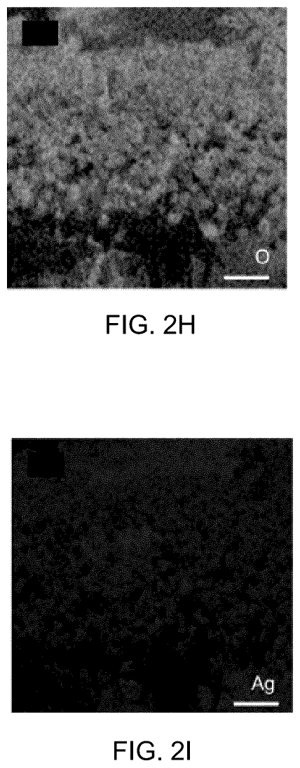

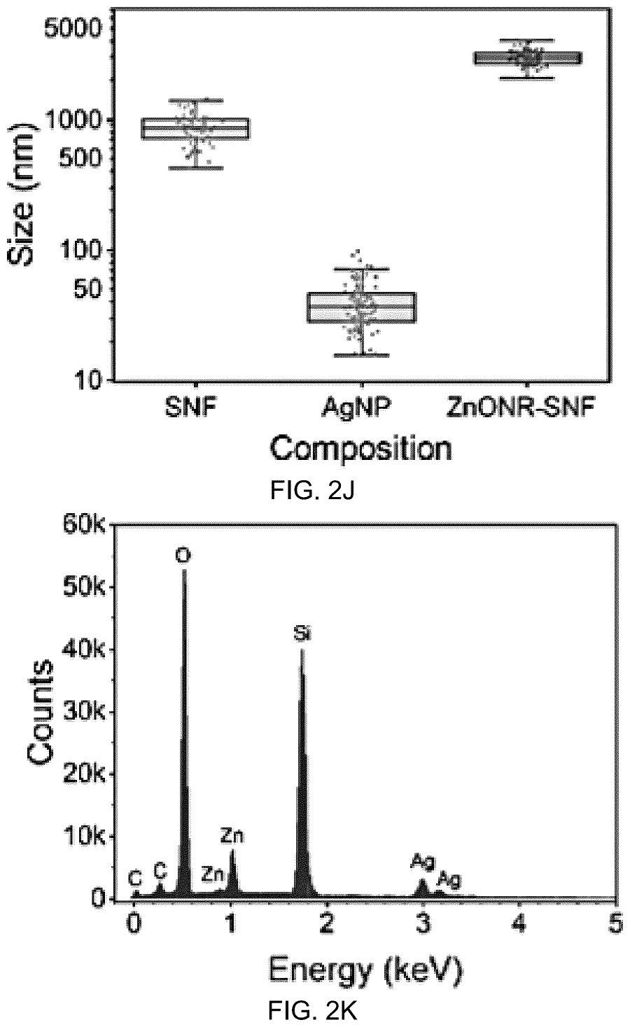

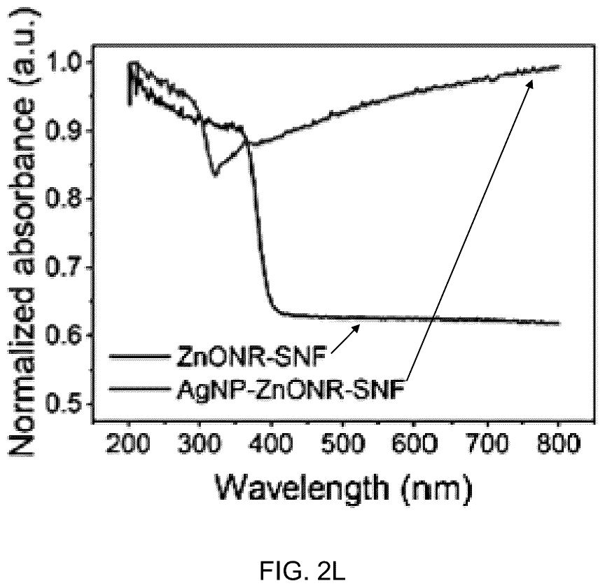

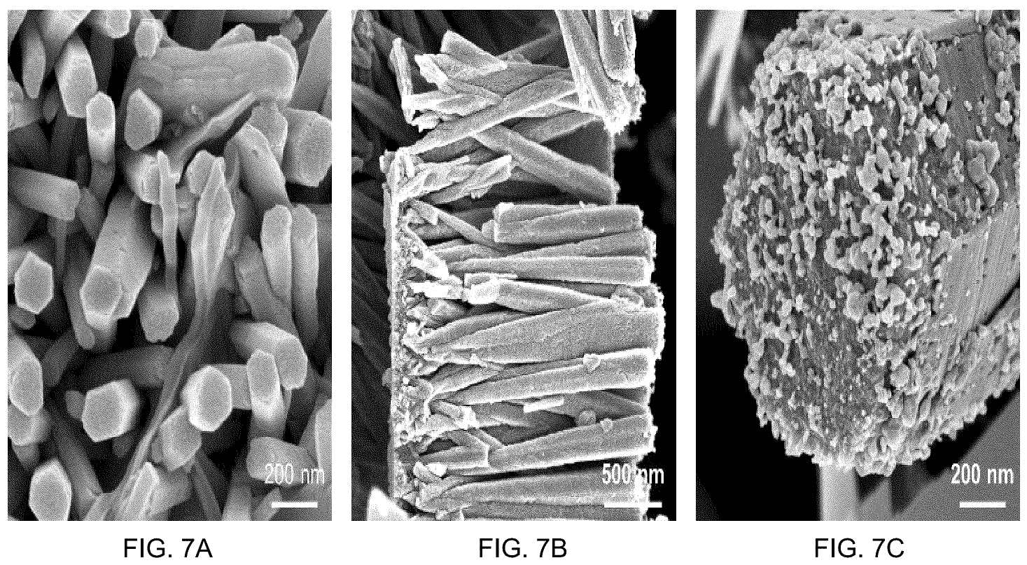

FIG. 2A depicts the synthesis process of electrospun SNF, ZnONR growth, and AgNP decoration. FIGS. 2B-2E are scanning electron microscopy (SEM) images demonstrating the structure of AgNP-ZnONR-SNF. Specifically, FIG. 2B displays a SEM image of SNF, FIGS. 2C and 2D display a SEM image of ZnONR-SNF, and FIG. 2E displays a SEM image of AgNP on the top of the ZnONR. Scale bars are 20 μm, 10 μm, 2 μm, and 100 nm, respectively. The AgNPs were distributed three-dimensionally, with some on the top of the ZnONRs and others on their side walls, while pure ZnONRs had a smooth surface, as shown FIGS. 7A-7C. Specifically, FIGS. 7A-7C display SEM images of top-view ZnONR (FIG. 7A), side-view ZnONR (FIG. 7B) and AgNP on the top of the ZnONR (FIG. 7C).

As shown in FIGS. 2F-2I, the energy dispersive spectrometry (EDS) chemical mapping confirms the uniform distribution of Zn, O, and Ag elements in the sample. FIG. 2F displays the EDS mapping of the AgNP-ZnO-SNF, including a SEM image, FIG. 2G displays the Zn element, FIG. 2H displays the 0 element, and FIG. 2I displays the Ag element. Scar bars are 3 μm.

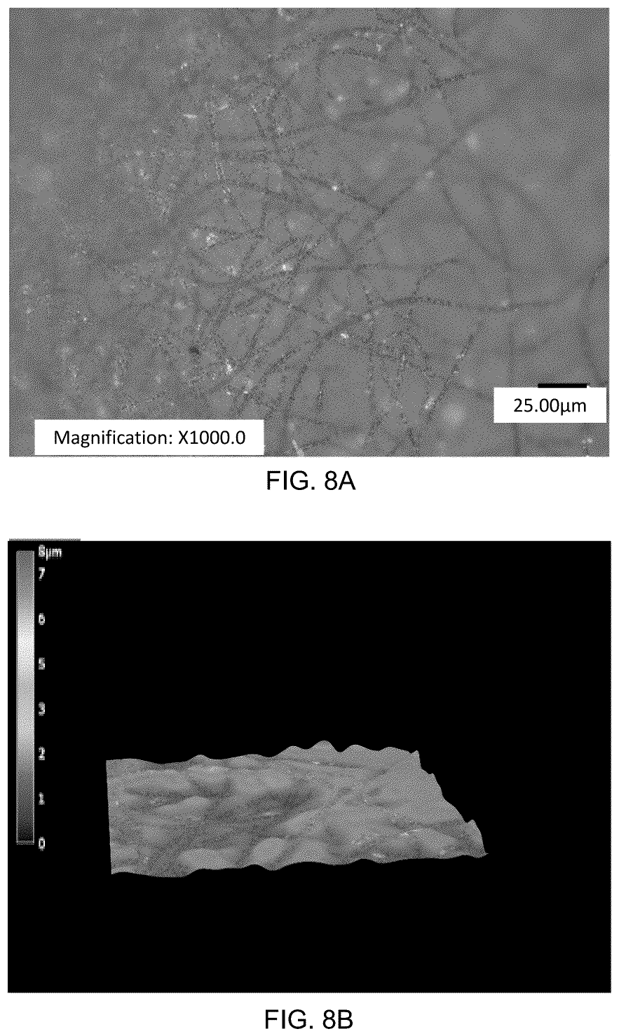

FIG. 2J displays the diameter distribution of the SNF, AgNP, and ZnONR-SNF in semi-logarithmic plot. Within each box, the horizontal lines denote the median values. The boxes extend from the 25^{th} to the 75^{th} percentile of each group's distribution of values. The vertical extending lines denote the most extreme values within the 1.5 interquartile range of the 25^{th} and 75^{th} percentile of each group. The dots denote observations. As shown, the average diameter of the SNF, AgNP and ZnO-SNF was 868 nm, 40 nm and 3 μm, respectively (FIG. 2J). The characteristic X-ray energy of the key elements, including Zn, Si, O, and Ag, were plotted and labeled in the energy spectrum as shown in FIG. 2K. The Ag element had its characteristic energies at 2.98 keV and 3.15 keV, the Zn element at 1.01 keV, the O element at 0.52 keV, and the Si element at 1.74 keV. Additionally, Optical images taken by Keyence 3D Microscope with high magnification revealed the fibrous and 3D structure of the AgNP-ZnONR-SNF thin film, as shown in FIGS. 8A and 8B. Specifically, FIG. 8A displays optical images of AgNP-ZnONR-SNF with 1000 magnification and FIG. 8B displays 3D reconstruction of the AgNP-ZnONR-SNF thin film. FIG. 2L exhibited the normalized UV-Visible optical absorption spectrum of the ZnONR-SNF and AgNP-ZnO-SNF thin films.

### Piezo-Catalytic and Photo-Catalytic Degradation of ZNONR-SNF

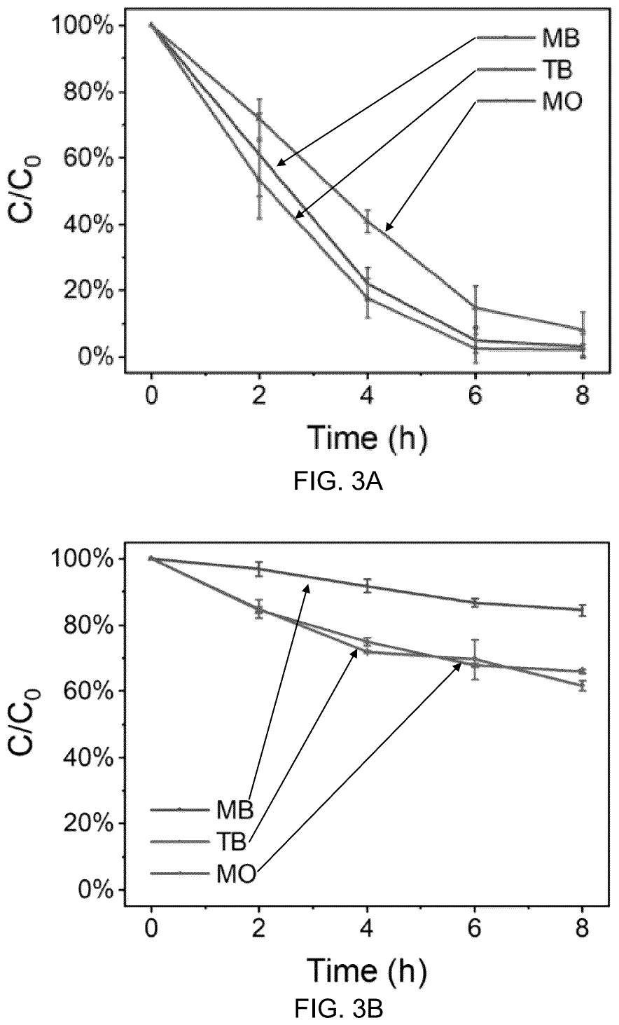

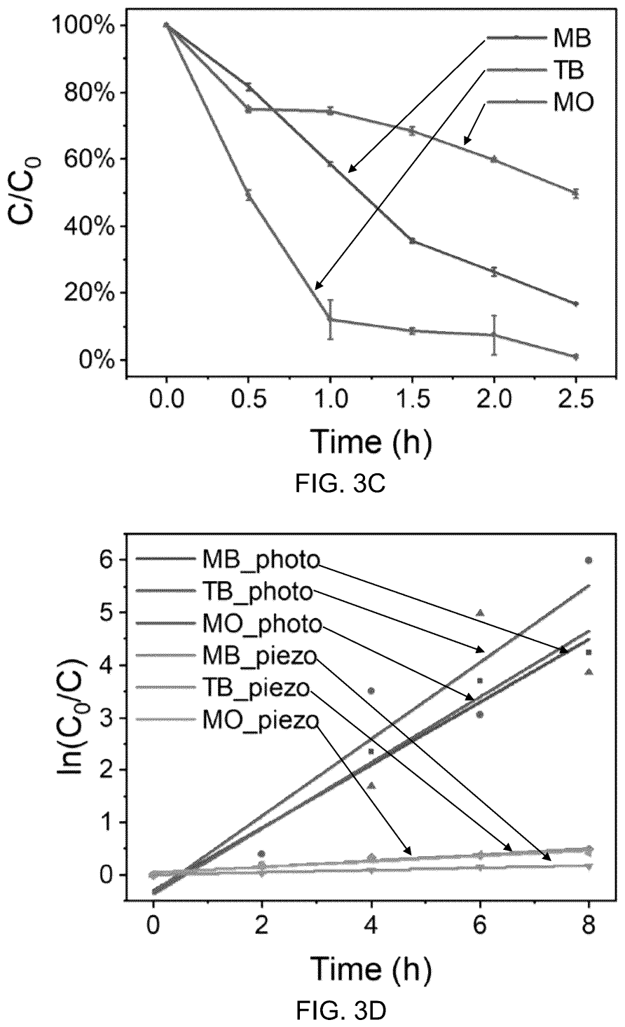

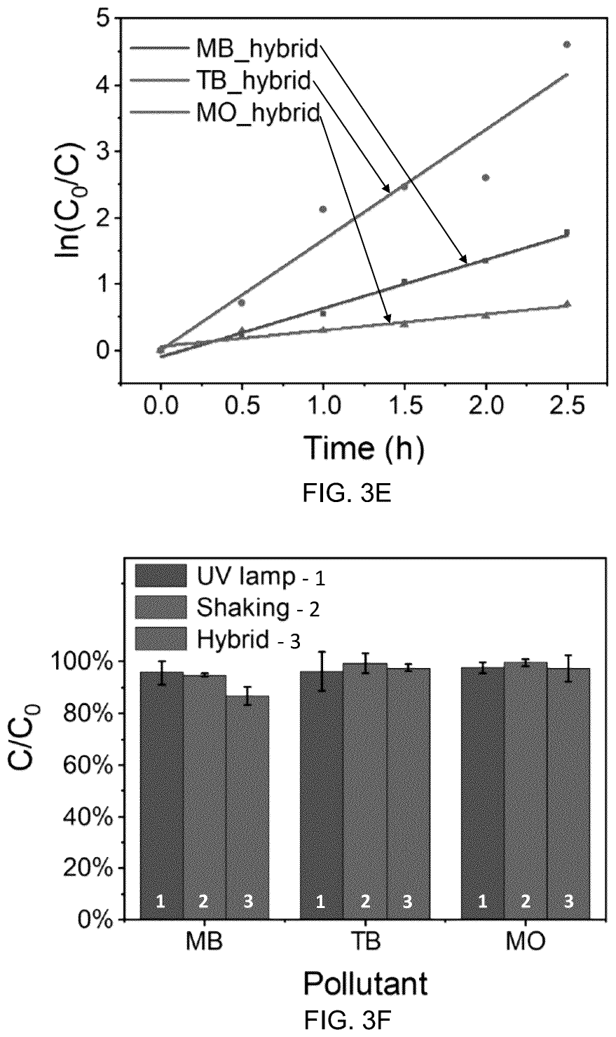

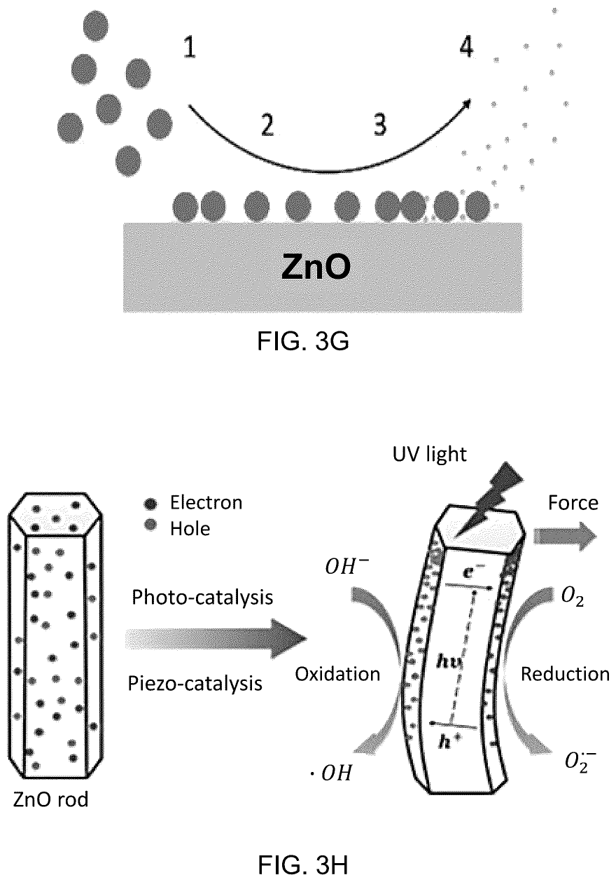

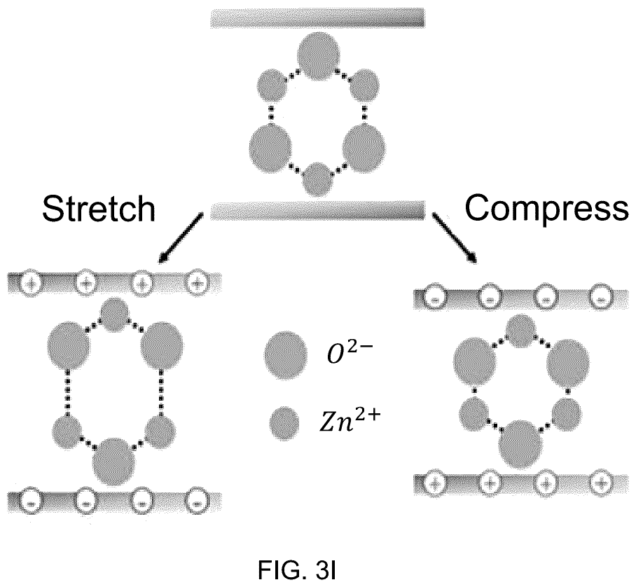

In this example, the piezo-catalytic and photo-catalytic activities of ZnONR-SNF thin films in the degradation of organic dyes (MB, TB, and MO) at an initial concentration of 10 μg/mL were investigated. Experiments were performed using an orbiting shaker and UV lamp. Control experiments demonstrated that solar UV irradiation and shaking without any photocatalyst is negligible, and the concentration of the dyes remains almost unchanged. When ZnONR-SNF thin films are present, a degradation efficiency greater than 98% was achieved against the three organic dyes after eight hours of exposure to UV light. FIGS. 3A-3C displays the change in concentration of these organic pollutants over time under different conditions. FIG. 3A displays the photocatalytic degradation results, FIG. 3B displays the piezo-catalytic degradation results, and FIG. 3C displays the hybrid degradation results.

Notably, it was found that the decline of the light absorbance intensity of the organic dyes at their characteristic peak wavelength was due to the cleavage of chromophore groups responsible for dye decoloration. Experiments were performed under sunlight and under a UV lamp to determine the photocatalysis degradation of MB, TB, and MO. The time interval was two hours, and the total irradiation time was eight hours.

Photocatalytic degradation efficiency for samples after two hours of sunlight exposure was even higher than under the UV lamp. FIGS. 3D and 3E display the rate constants (k) for photocatalytic degradation, confirming the excellent photocatalytic property of ZnONR-SNF thin films. Specifically. FIG. 3D displays the photo- and piezo-catalytic degradation kinetic curves of dye solutions catalyzed by the ZnONR-SNF, and FIG. 3E displays the hybrid degradation kinetic curves of dye solutions catalyzed by the ZnONR-SNF. The degradation outcomes of the control groups lacking the ZnONR-SNF thin films are illustrated in FIG. 3F. The shown error bars represent the standard deviation. In these groups, only a marginal decrease in dye concentrations was observed.

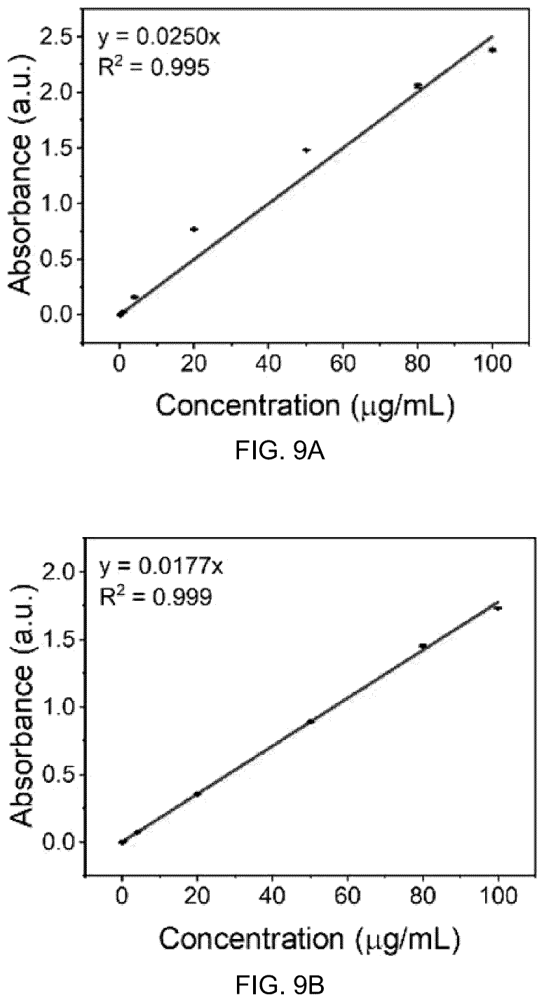

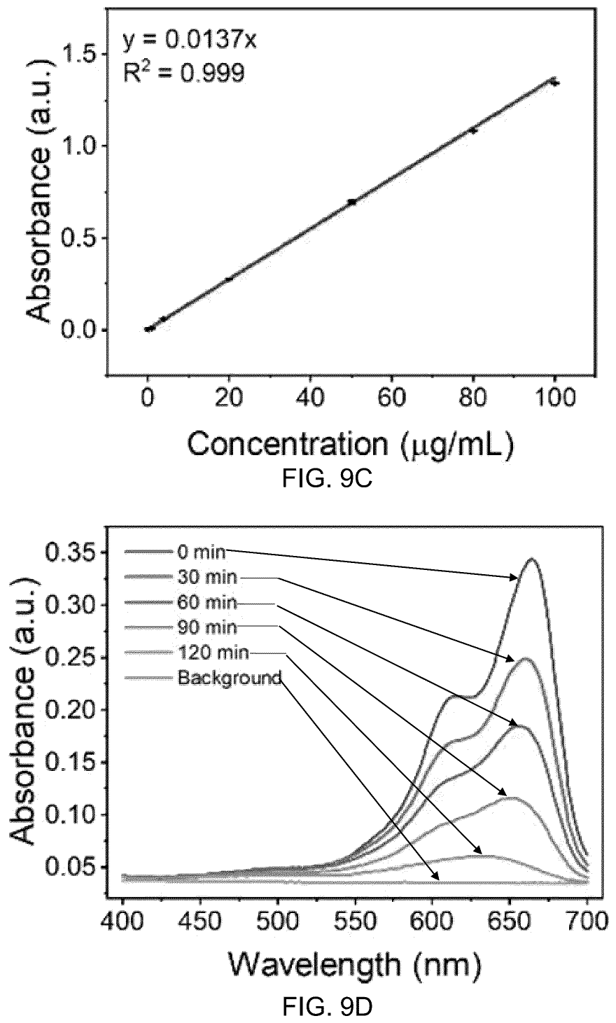

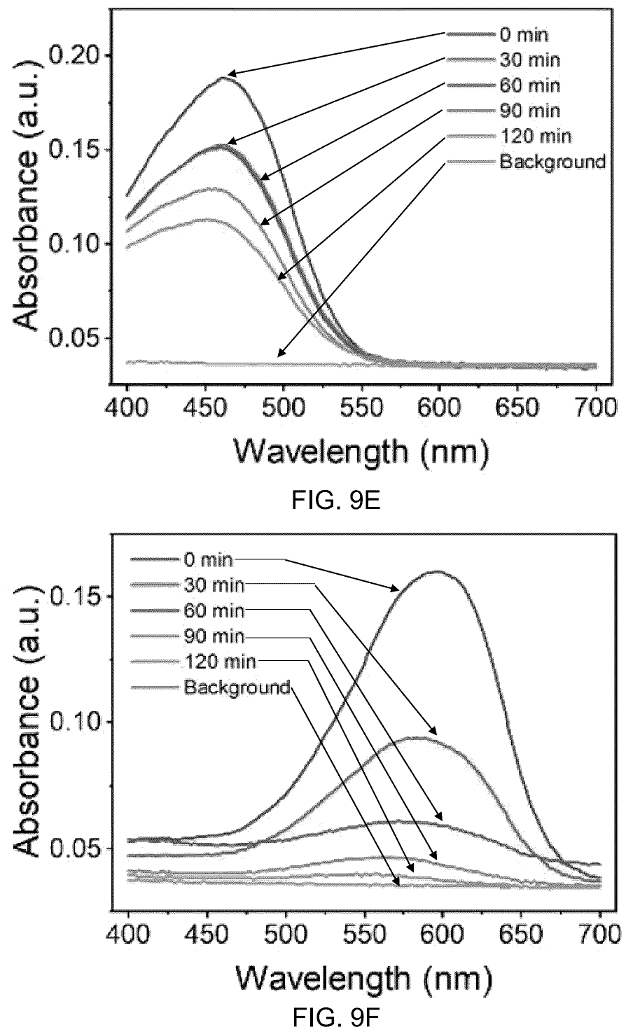

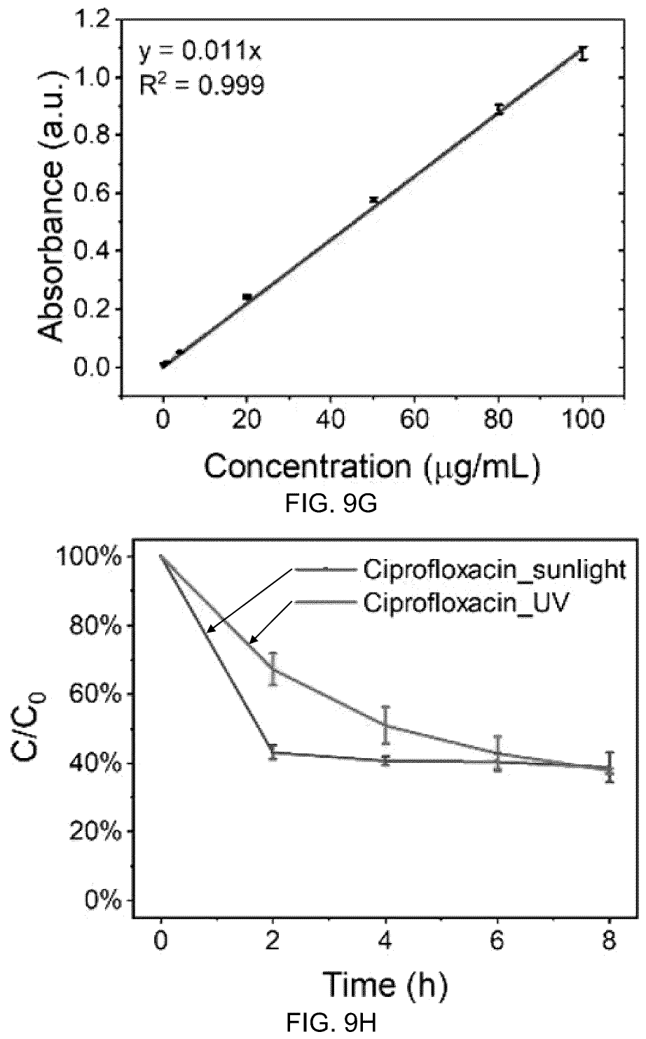

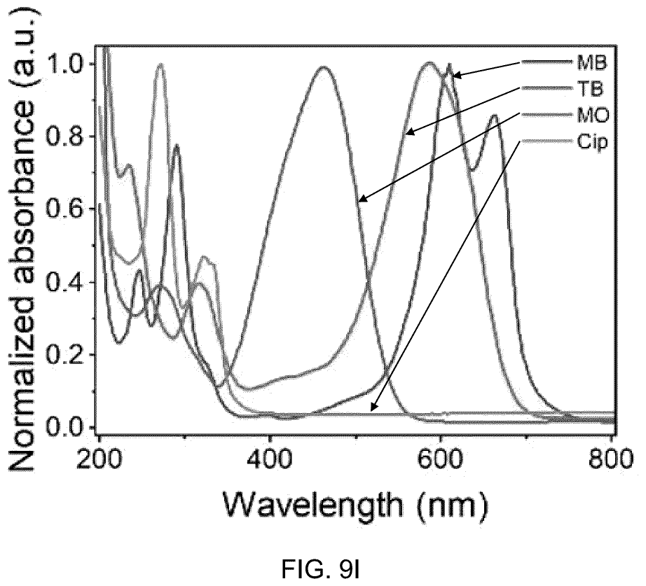

In addition to organic dyes, the photocatalysis degradation against antibiotic (Cip) was also studied under both UV light and sunlight, as shown in FIGS. 9A-9I. Specifically, FIGS. 9A-9I display UV-Visible absorbance curves and calibration curves of different molecules. The concentration-absorbance calibration curves of MB are shown in FIG. 9A, the concentration-absorbance calibration curves of MO are shown in FIG. 9B, and the concentration-absorbance calibration curves of TB are shown in FIG. 9C. UV-Visible absorbance curves of MB are shown in FIG. 9D, UV-Visible absorbance curves of MO are shown in FIG. 9E, and UV-Visible absorbance curves of TB are shown in FIG. 9F under different UV irradiation time. FIG. 9G displays the concentration-absorbance calibration curve of Cip. FIG. 9H displays the photocatalytic degradation results of Cip under UV lamp and sunlight. FIG. 9I displays the normalized UV-Visible absorbance curves showing the characteristic peaks of different molecules.

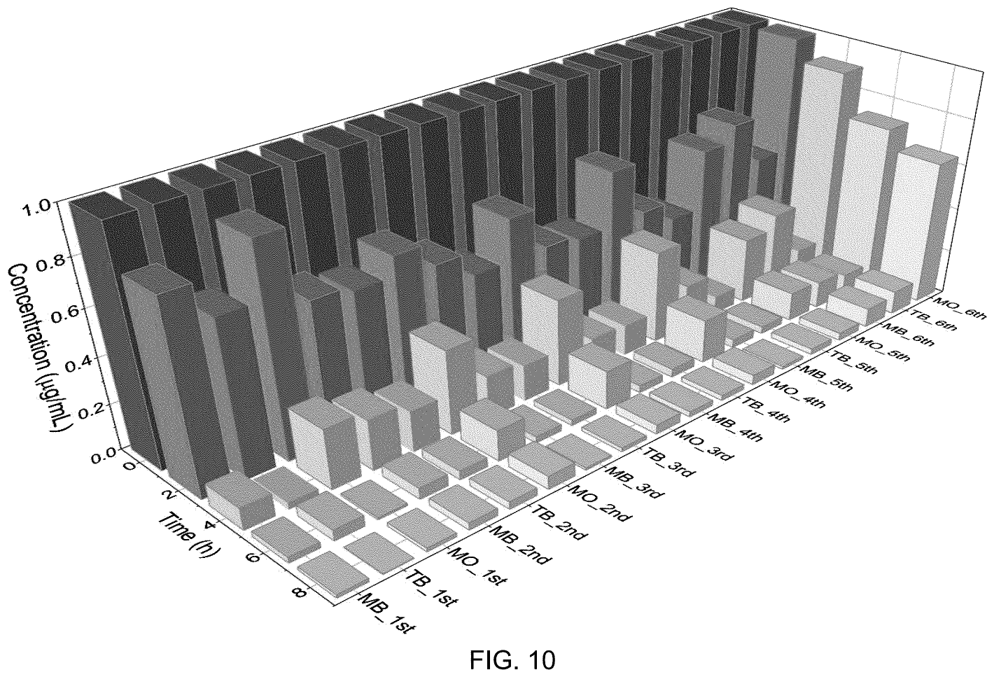

The reusability of the ZnONR-SNF thin film may also be an essential factor in its efficiency in the photocatalytic degradation. The same ZnONR-SNF samples were used to degrade MB, TB, and MO under UV lamp and dried in the oven at 50° C. for six cycles. The photocatalytic efficiency of ZnONR-SNF thin films remained high for five cycles, as shown in FIG. 10. Specifically, FIG. 10 displays the reusability test of the photocatalytic property of ZnONR-SNF thin film against MB, TB, and MO.

The photocatalytic degradation against organic molecules can be explained by the physical and optical properties of ZnO (FIGS. 3G and 3H) as follows: first organic pollutants diffuse from the liquid phase and are absorbed onto the surface of ZnONR. Then ZnONR is irradiated by the UV light with energy larger than its bandgap energy, promoting electrons (e⁻) from valence band (VB) to conduction band (CB) and leave holes (h⁺) in the VB. Next, the photogenerated electron-hole (e⁻/h⁺) pairs can migrate to the ZnONR surface, reacting with water and hydroxide ions (by h⁺) and oxygen (by e⁻) to generate reactive oxygen species (ROS) including hydroxyl radical (OH) and superoxide anion (O₁^{·-}). Finally, the ROS can directly oxidize organic pollutant molecules.

The piezo-catalytic activity of ZnONR-SNF thin film on degrading organic dye solution is shown in FIG. 3I. The catalyst was stimulated through orbiting shaking using a shaker motor with a rating output of 13.5 W. After eight hours of reaction, the piezo-catalytic efficiency against MB, TB, and MO reached 15.5%, 38.4% and 34.1%, respectively. The deformation of ZnONR under the influence of mechanical force generated by the flowing water created a strain field, resulting in the outer side being stretched and the inner side being compressed (FIG. 3H). This deformation led to the generation of an electric field along the ZnONR, which induced a piezoelectric potential and surface charge accumulation on the opposite surface. This allowed e⁻/h⁺ to migrate to the ZnONR surface and triggered subsequent reactions similar to the photocatalytic degradation process. Additionally, the piezoelectric potential also contributed to the adsorption of charged organic molecules.

In hybrid experiments, both UV lamp and shakers were used which led to the degradation of 96.3% of MB, 66.4% of TB, and 34.8% of MO after two hours of irradiation, which was greater than the two catalytic activities alone. The improved degradation efficiency can be explained by the flowing water and semiconducting nature of ZnONR. The circulating water flow created by the orbiting shaker not only deflects ZnONR to create piezoelectric potential between the opposite surface, but also promotes the adsorption of organic molecules. Moreover, the relative displacement of the Zn²⁺ cations regarding the O²⁻ anions in the wurtzite crystal structure (FIG. 3I) generates a piezoelectric potential along the nanorod direction when the ZnONR is bent. This potential difference maintains as long as the strain exists and creates a potential difference between the compressed and the stretched side surface. Correspondingly, the recombination of photo-induced e⁻/h⁺ pairs inside the ZnONR will be impeded, leading to more efficient carrier separation. Eventually, more redox reaction occurs on the surface of ZnONR, to improve the photocatalytic degradation.

### SERS Detection

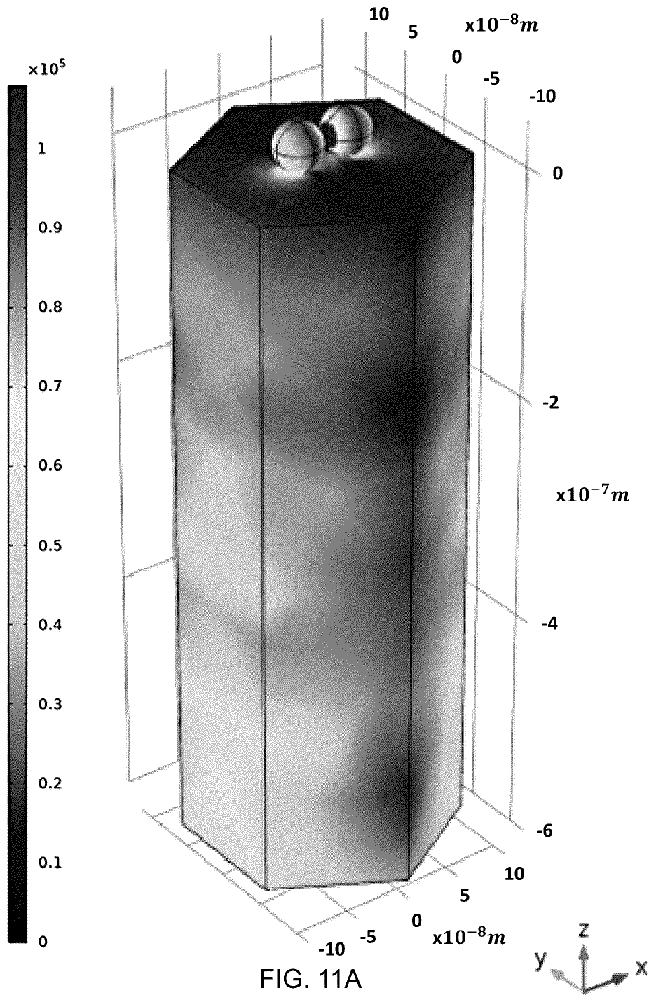

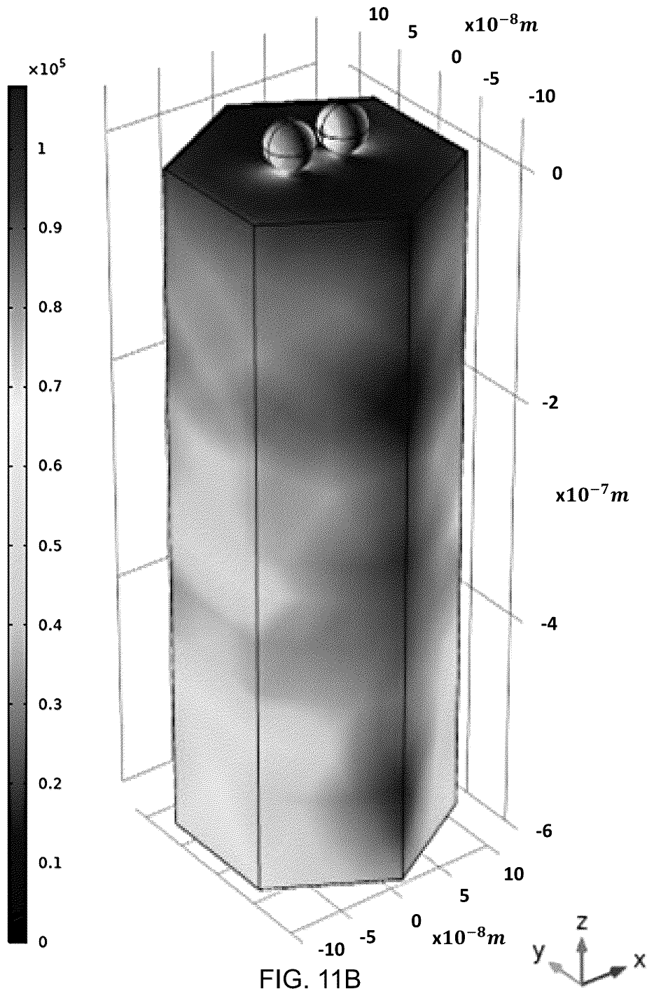

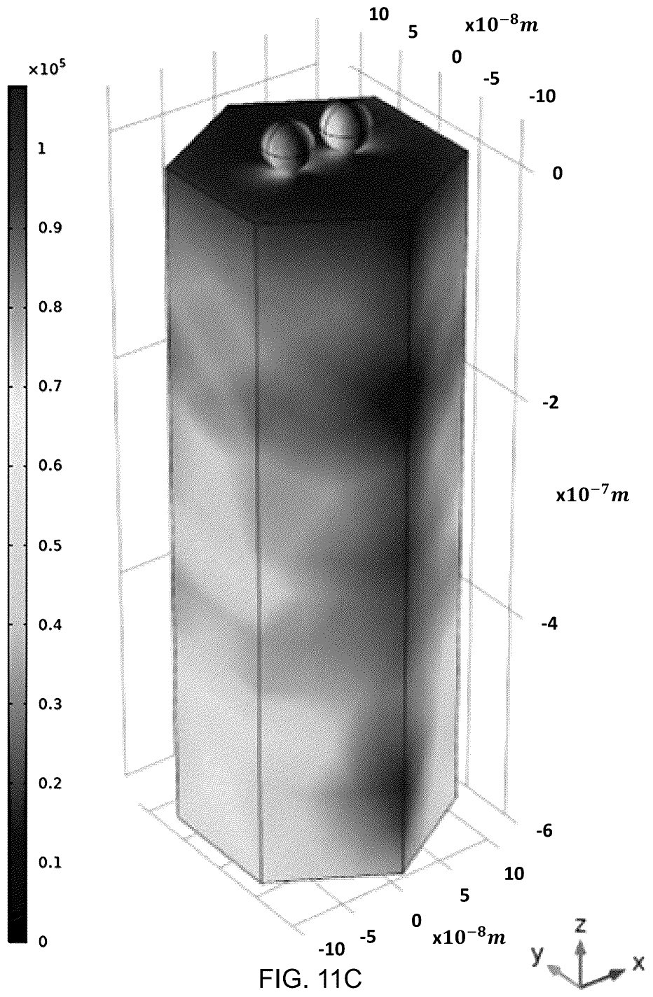

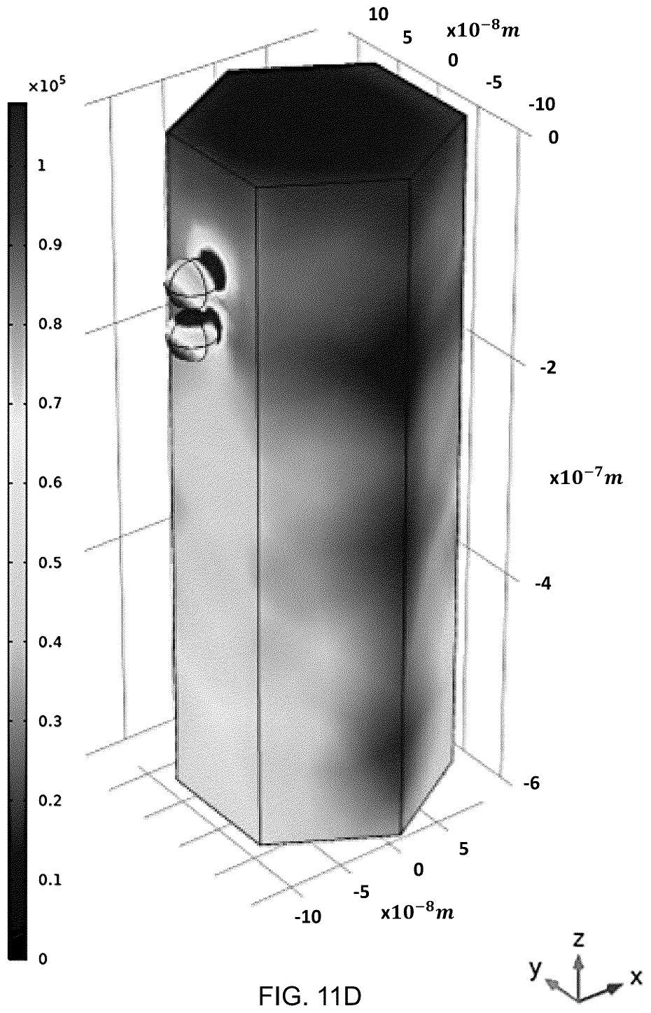

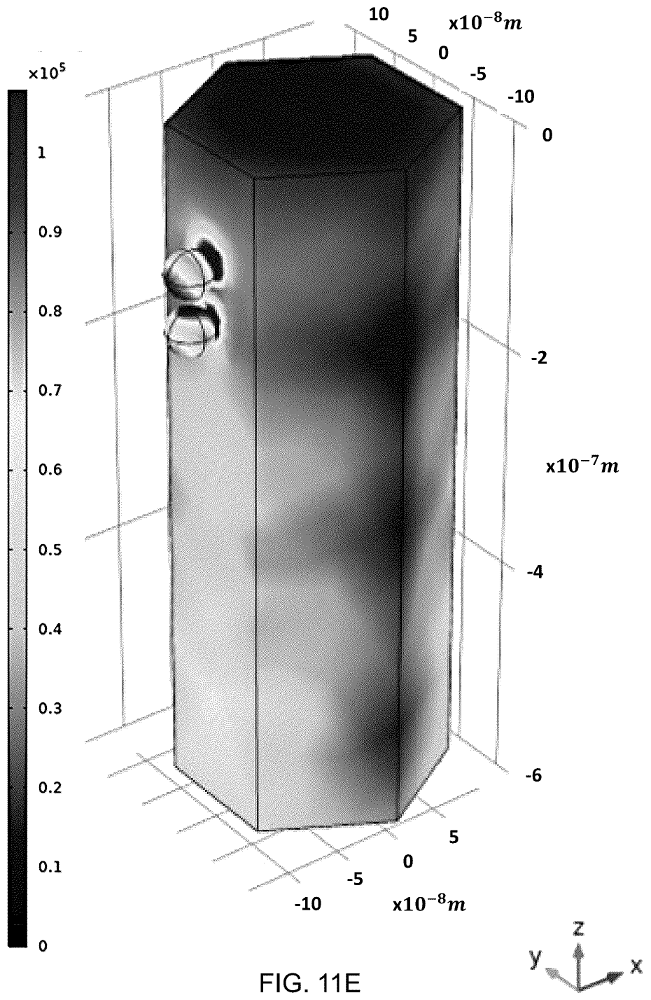

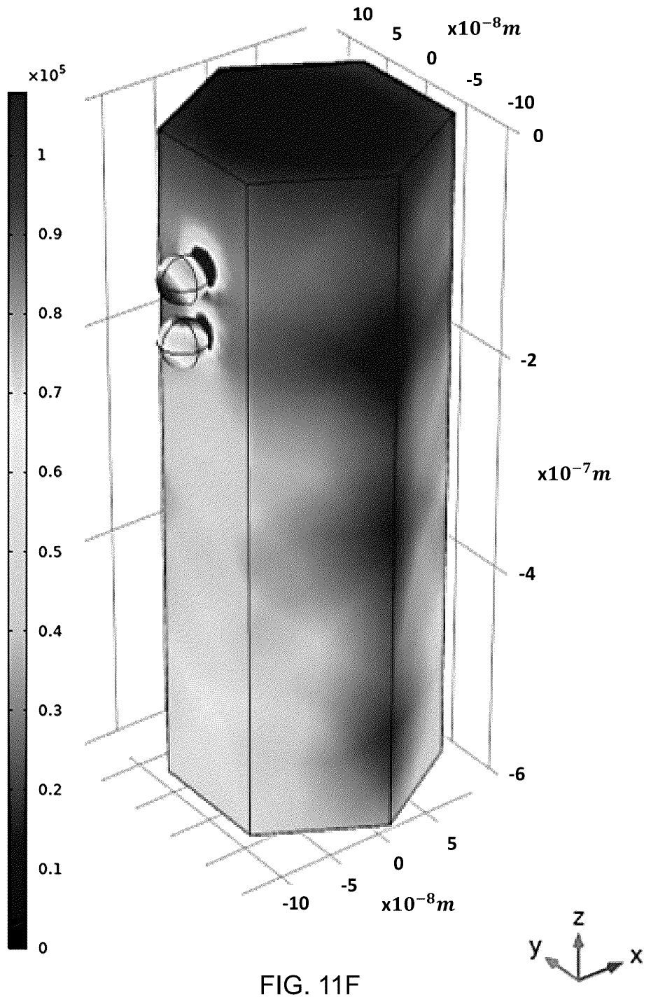

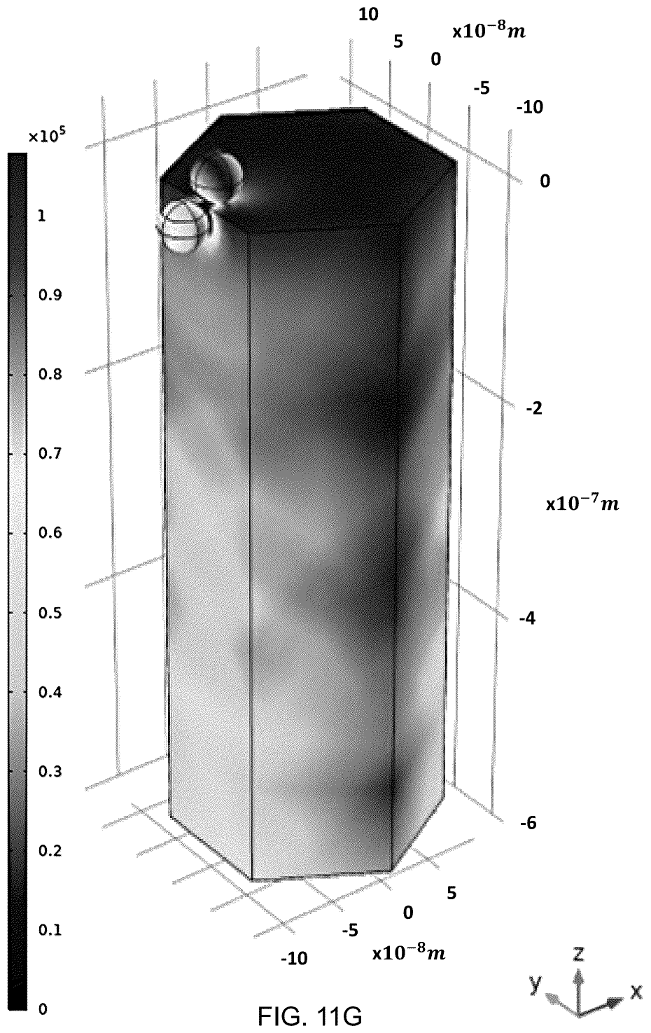

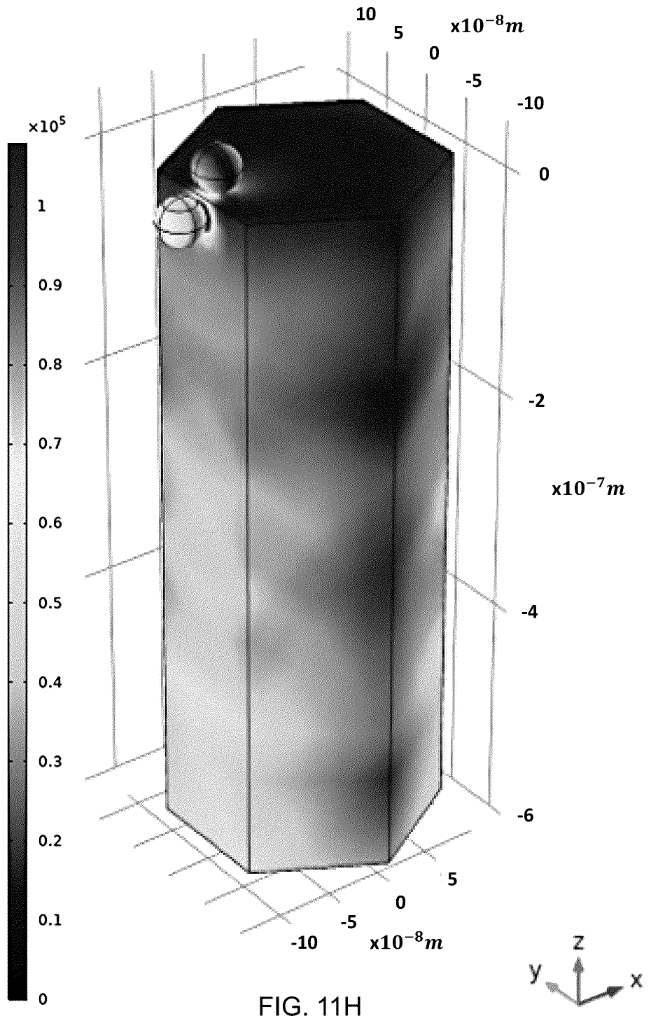

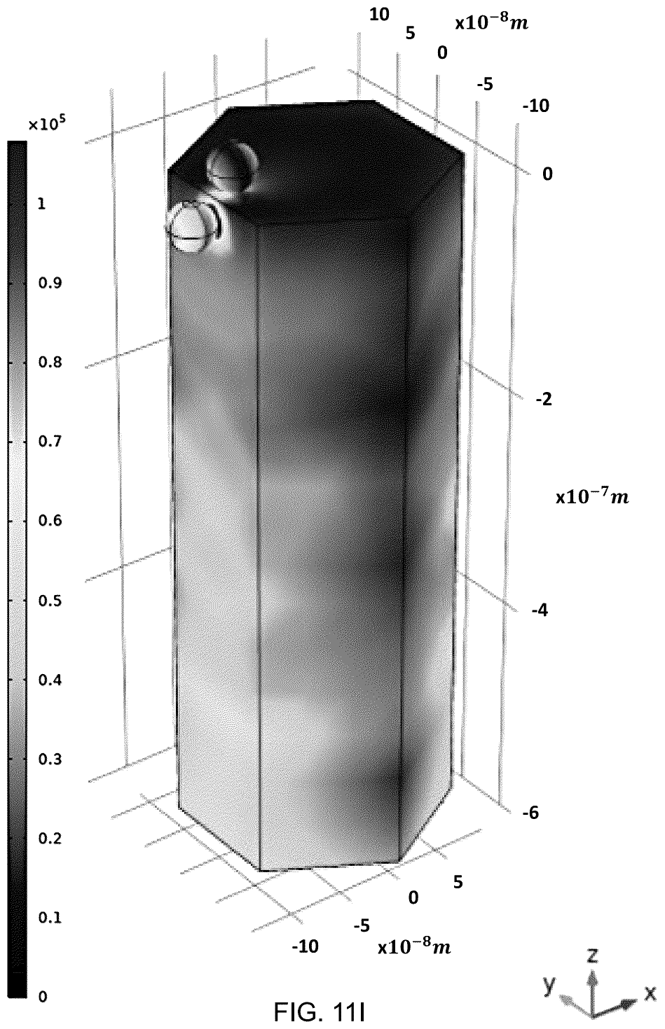

Raman spectroscopy provides a label-free and rapid tool for sensing organic molecules. A SERS approach was used in this example to quantitatively measure the concentration of an organic dye and an antibiotic. Before conducting the Raman experiments, the enhancement of localized surface plasmonic resonance was simulated using the finite element method (FEM) by COMSOL. Three scenarios of AgNP placement were observed in the SEM images: on the top side, on the side wall, and at the edge of ZnONR, as shown in FIGS. 11A-11I. Specifically, FIGS. 11A-11I display a simulation of the localized surface plasmon enabled electrical field enhancement of the Ag nanoparticle dimer at different location under 532 nm light. The Ag nanoparticle dimer of 40 nm diameter on the top surface of ZnONR with gap of 5 nm is shown in FIG. 11A, of 10 nm is shown in FIG. 11B, and of 15 nm is shown in FIG. 11C. Ag nanoparticle dimer of 40 nm diameter on the side surface of ZnONR with gap of 5 nm is shown in FIG. 11D, of 10 nm is shown in FIG. 11E, and of 15 nm is shown in FIG. 11F. Ag nanoparticle dimer of 40 nm diameter at the edge of ZnONR with gap of 5 nm is shown in FIG. 11G, of 10 nm is shown in FIG. 11H, and of 15 nm is shown in FIG. 11I.

It was observed that when the AgNPs are in close proximity and coupling with incident light, a plasmonic “hotspot” region is created where localized electromagnetic field is dramatically enhanced occurs. This enhanced electromagnetic field can amplify SERS intensity to the fourth power of field enhancement, and the “hotspot” sites created by AgNP dimers generated more than 50% of the total SERS signal with only 1% of total surface area.

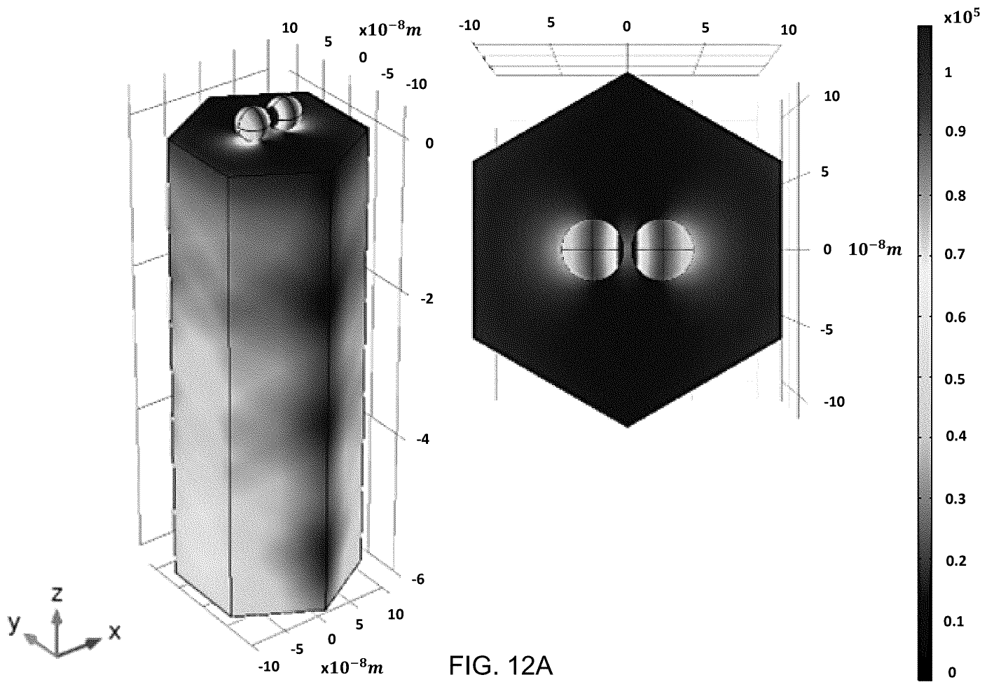

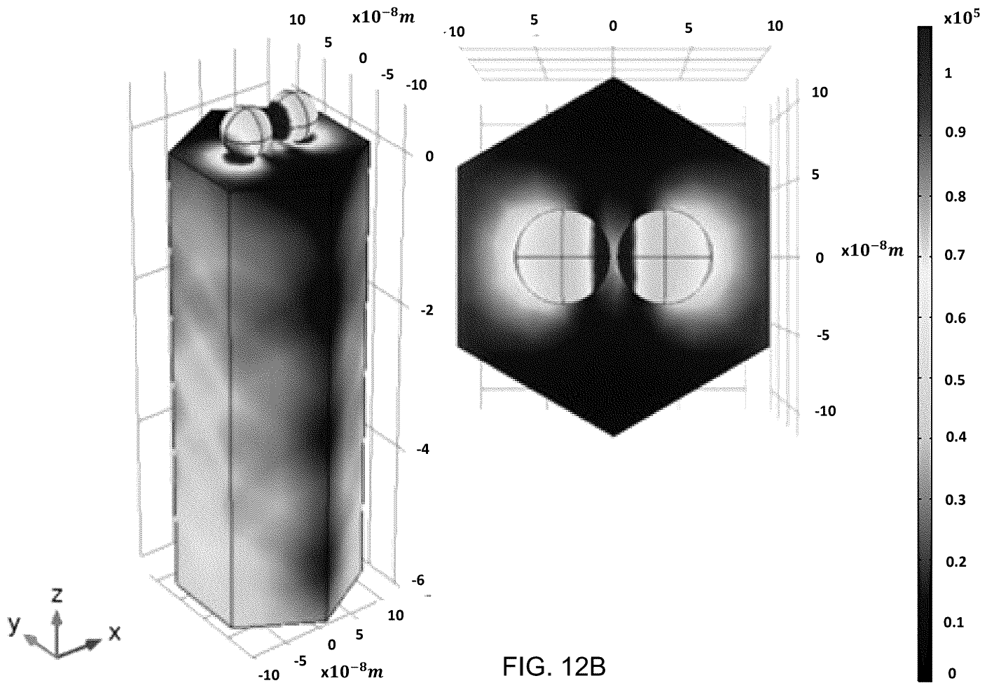

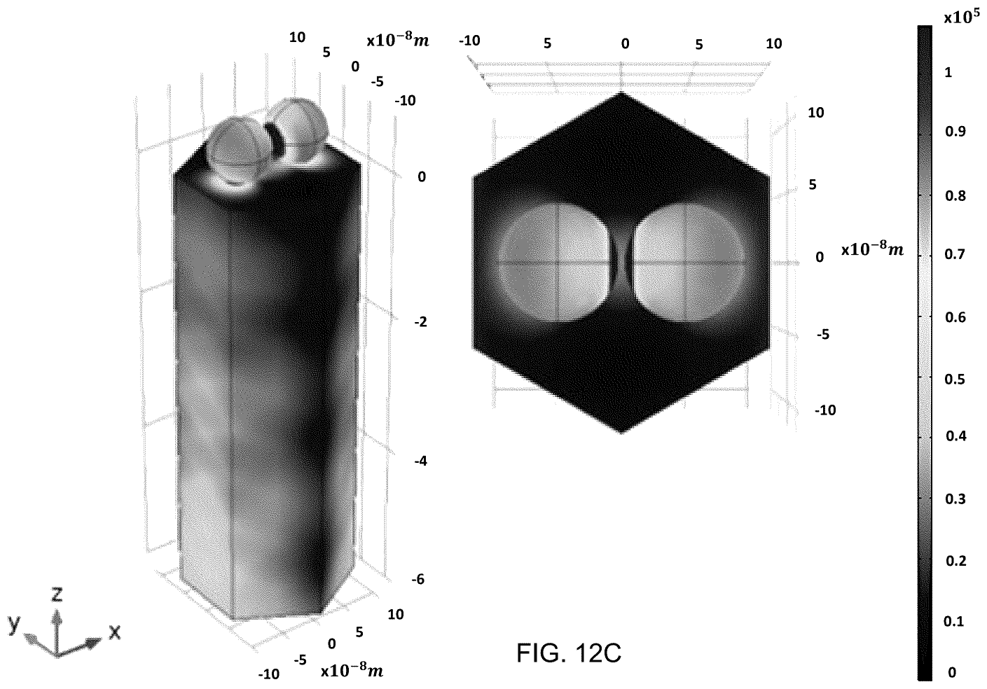

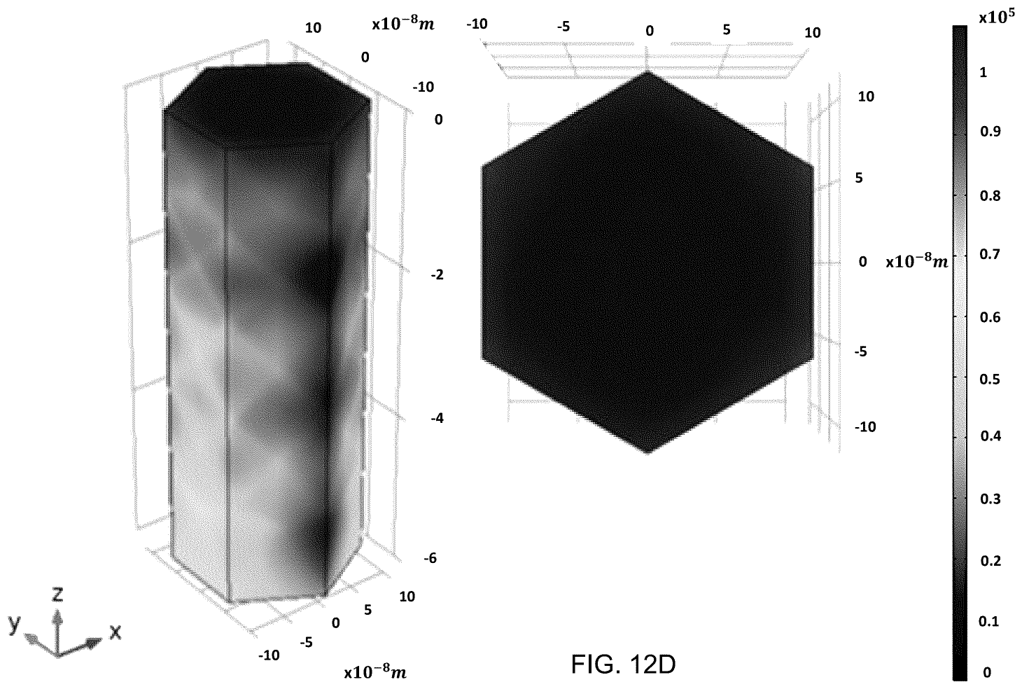

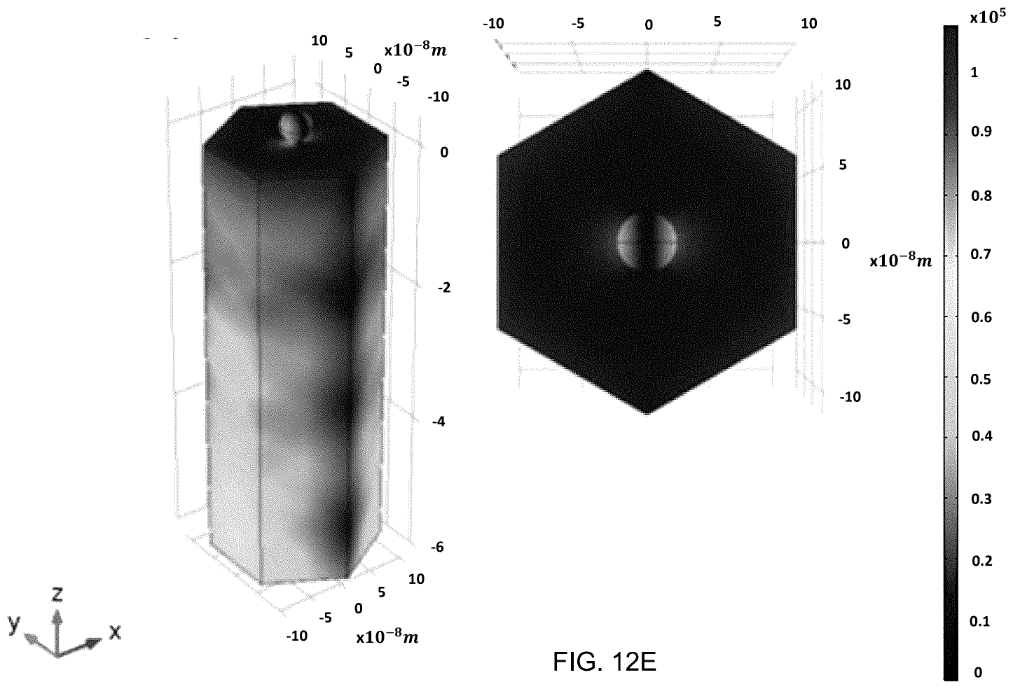

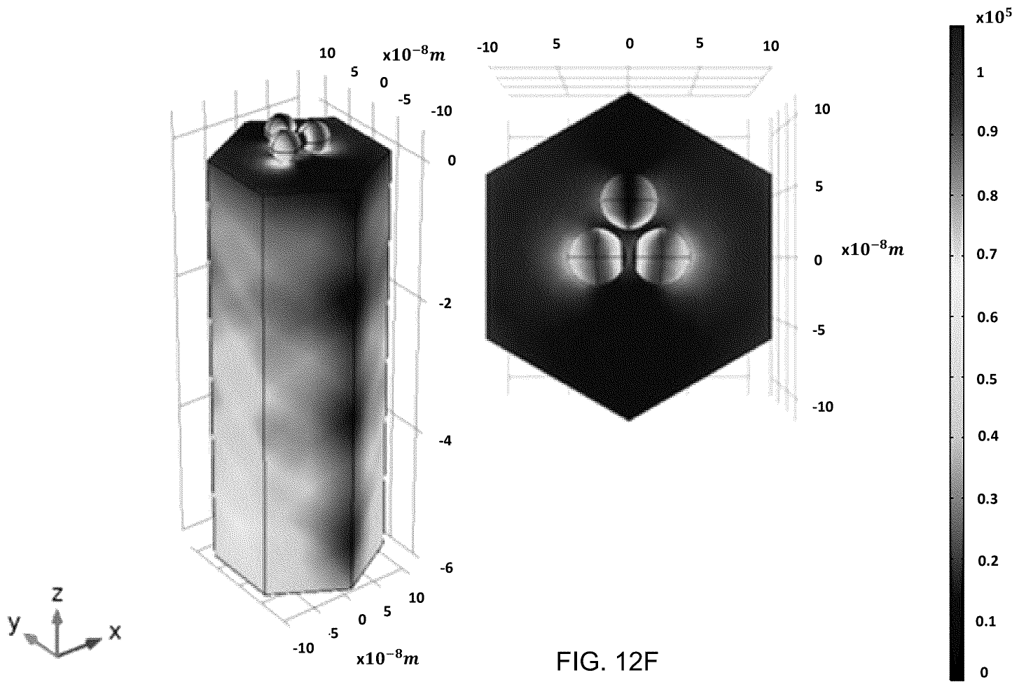

This example aims to leverage ZnONR. This multifunctional material serves not only as a photo-catalyst and piezo-catalyst, but also functions as a stable support for decorating with AgNPs, thereby enhancing SERS sensing uniformity. Additionally, AgNP trimer structure on the top of ZnONR was found to enhance the field, as shown in FIGS. 12A-12F, which aligns with the experiment observation. Specifically, FIGS. 12A-12F displays simulation of the localized surface plasmon enabled electrical field enhancement of the Ag nanoparticle with different diameter and pair number under 532 nm light. An Ag nanoparticle dimer of 40 nm diameter is shown in FIG. 12A, of 60 nm diameter is shown in FIG. 12C, and of 80 nm diameter is shown in FIG. 12E on the top surface of ZnONR with 5 nm gap.

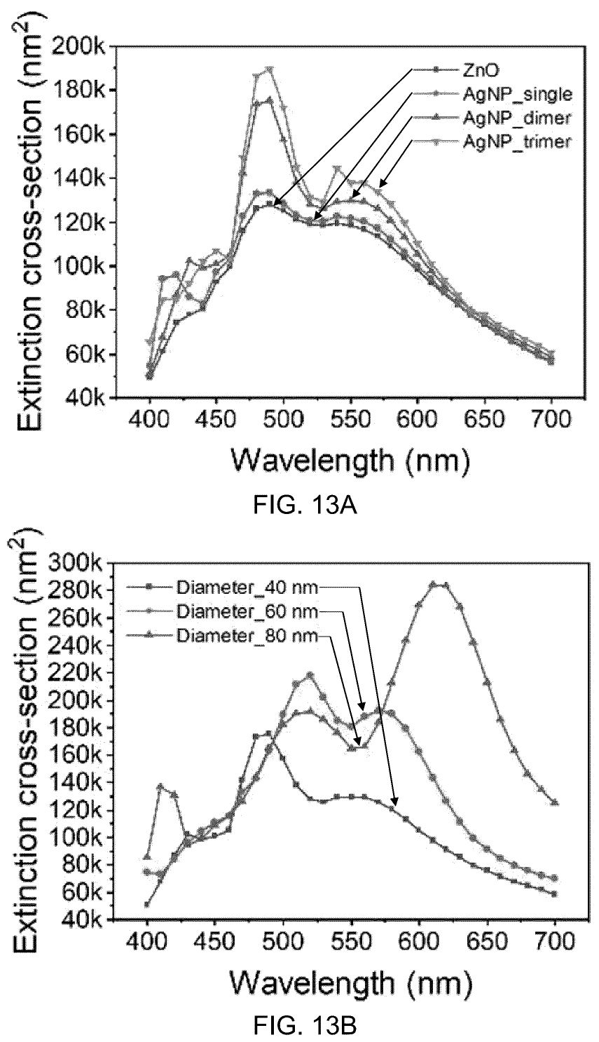

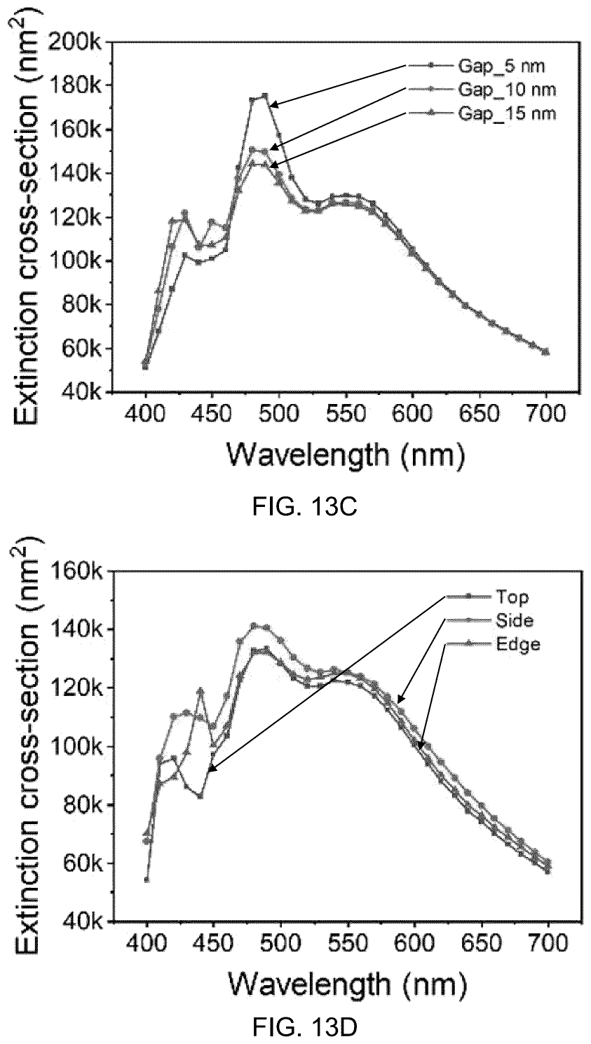

According to the simulation results of this example, the diameter and the gap between the AgNPs may affect the enhancement, while the locations may have limited effect, as shown in FIGS. 13A-13D. As most of the peaks of the extinction cross-section area for all AgNP arrangements lay in the range from 475 nm to 525 nm, a 532 nm laser was chosen as the excitation source.

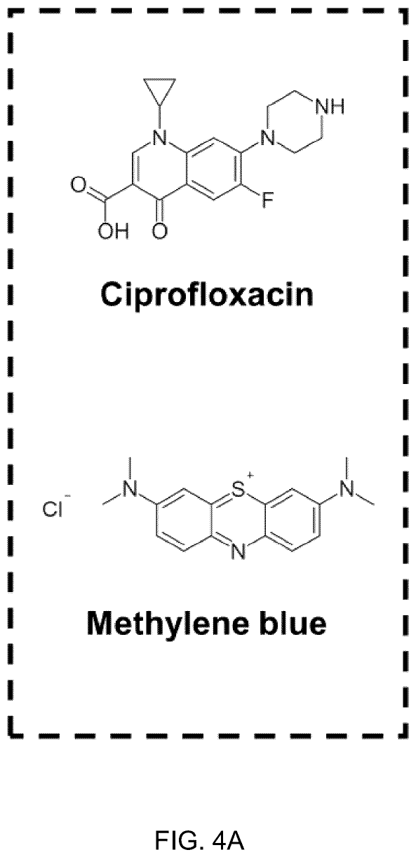

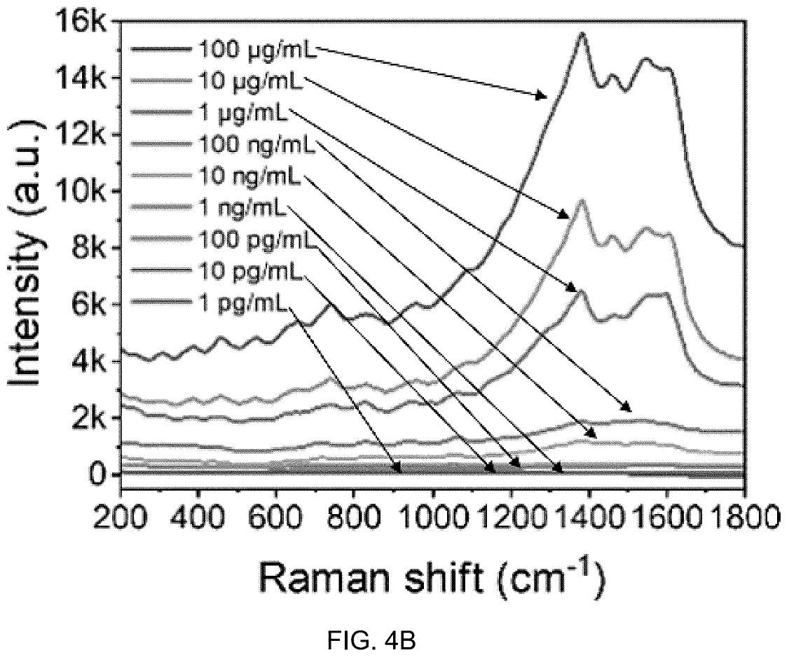

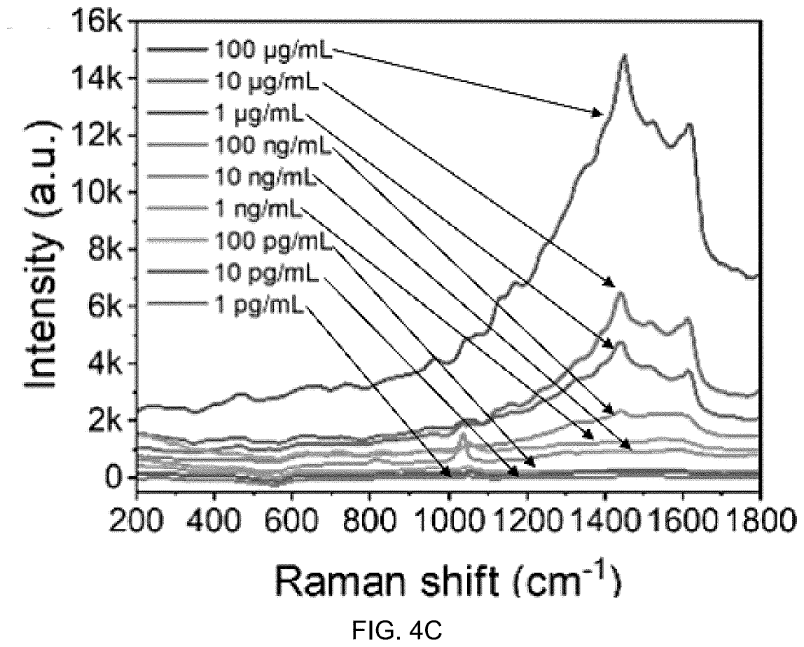

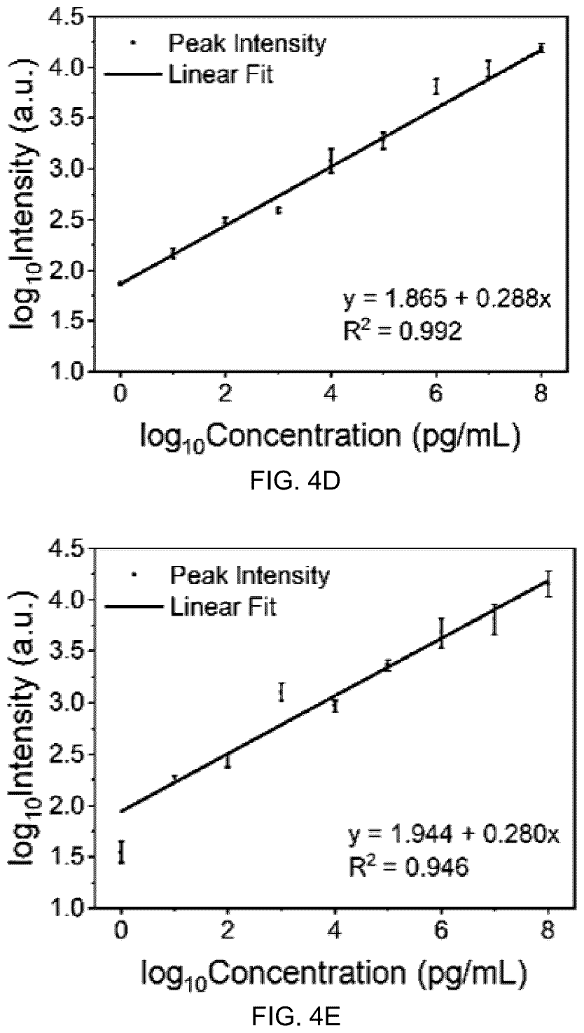

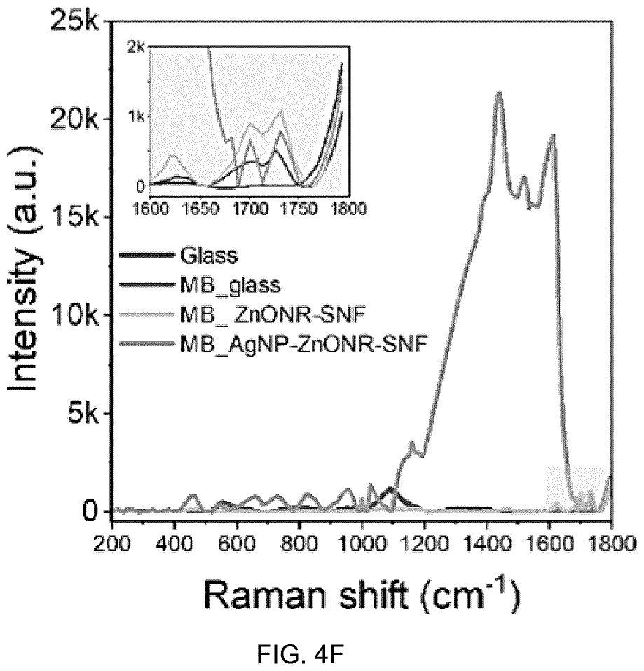

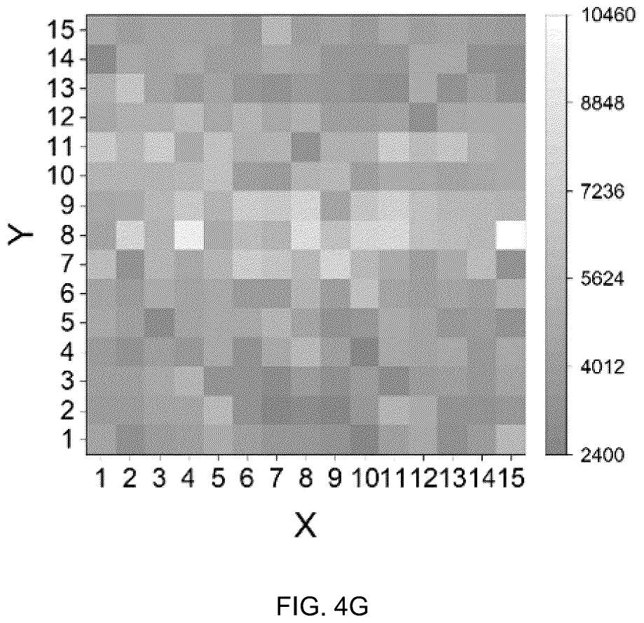

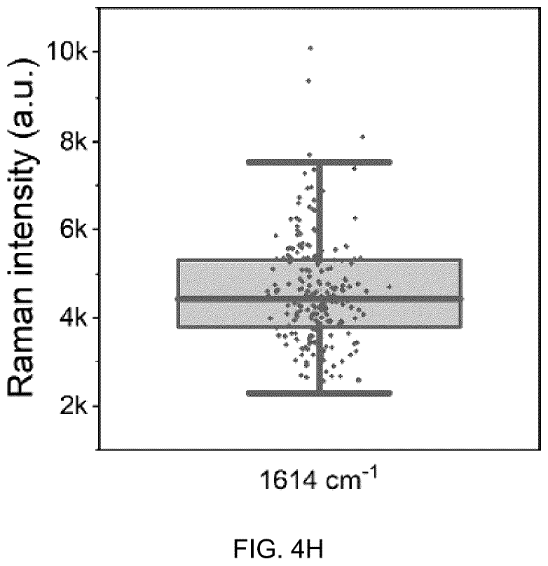

In the Raman experiments performed in this example, MB and Cip, the chemical structures shown in FIG. 4A, were used as demonstrations. In the test, 10 μL of the sample solution were dropped on the AgNPZnONR-SNF chip that had a size of about 5 mm×5 mm. FIG. 4B and FIG. 4C show the raw Raman spectrum of the Cip and MB samples, respectively, at concentrations ranging from 100 μg/mL to 1 pg/mL. The most distinguishable characteristic peaks of the Cip are at 1382 cm⁻¹, 1465 cm⁻¹, 1605 cm⁻¹, and 1548 cm⁻¹, and MB at 1437 cm⁻¹ and 1614 cm⁻¹. As shown, there is a general trend of concentration of the samples being proportional to the intensity of the Raman signal. Therefore, the intensity of the highest peaks of the MB (1437 cm⁻¹) and Cip (1382 cm⁻¹) dyes were plotted against their concentrations as shown in FIG. 4D and FIG. 4E, respectively, on a log₁₀ scale. MB and Cip calibration curves show high linearity of R²=0.992 and R²=0.946, respectively, demonstrating the functionality of embodiments of the present disclosure as a sensor that can quantitatively monitor the concentration of organic dyes and antibiotics. Specifically, FIG. 4D displays the Raman intensity versus the concentration curve of Cip (in log₁₀ scale), and FIG. 4E displays the Raman intensity versus the concentration curve of MB (in log₁₀ scale). Error bars represent the standard deviation.

To demonstrate the signal enhancement from embodiments of the AgNP-ZnONR-SNF chip, Raman signals on various substrates were measured, including a pristine glass substrate with no specimen, a glass substrate with a drop (10 μL) of MB, ZnONR-SNF with a drop of MB, and AgNP-ZnONR-SNF with a drop of MB solution (FIG. 4F). Specifically, FIG. 4F displays the SERS enhancement of MB dye (1 μg/mL) on different substrates. The figure shows the zoomed area between 1600 cm⁻¹ and 1800 cm⁻¹. The intensity at the 1437 cm⁻¹ peaks of MB on the AgNPZnONR-SNF, ZnONR-SNF and the glass substrate were 19861.9, 62.1 and 18.8, respectively. The results demonstrated an increase in intensity when using the AgNP-ZnONR-SNF substrate as compared to the glass substrate. Specifically, the intensity from the ZnONR-SNF substrate was 3.3 times higher than that from the glass substrate, and the intensity from the AgNPZnONR-SNF sensor was 1056 times higher than that from the glass substrate. The inset of FIG. 4F shows the spectrum in the 1600 cm⁻¹ to 1800 cm⁻¹ range, which contains two other characteristic peaks of MB at 1700 cm⁻¹ and 1732 cm⁻¹. The intensities of these peaks from the AgNP-ZnONR-SNF were 645.3 and 778.2, respectively, which is about 1.8 times those from the glass substrate. These results indicated that the SERS enhancement may mainly occur in the range of 1050 cm⁻¹-1650 cm⁻¹, where the enhancement is up to 1056-fold. Enhancement outside this range is less than 2-fold, and various factors, such as the particle material (e.g., Ag, Au, or other noble metals) and the geometry of the nanomaterials (e.g., particle size), determine the major enhancement range. Therefore, to achieve a large enhancement in a wider range of spectrum, multiple types of nanoparticles and various sizes may be used to decorate the ZnONR. Moreover, it was found that the AgNP-ZnONR-SNF chip exhibited relatively consistent and uniform sensing performance on its surface.

A color map of the signal intensity of the highest peak (1614 cm⁻¹) of MB dye was plotted (15×15 data points on an area of 400×400 μm²) to represent the magnitude of the intensity, as shown in FIG. 4G. Specifically, FIG. 4G displays the Raman signal mapping (10×10 data points on an area of 400×400 μm²) of the 1614 cm⁻¹ characteristic peak of the MB dye showing uniform intensity across the AgNP-ZnONR-SNF chip. Microscale Raman mapping analysis revealed that the magnitude of the signal intensity was generally consistent across this testing area, indicating a uniform sensing ability across the chip. The box chart of FIG. 4H shows the distribution of the Raman signal intensity for the characteristic peak at 1614 cm⁻¹ of MB. Specifically, FIG. 4H displays the distribution of Raman intensity. Within the box, horizontal lines denotes the median value. The box extends from the 25th to the 75th percentile of the group's distribution of values. The vertical extending lines denote the most extreme values within 1.5 interquartile range of the 25th and 75th percentile of the group. The dots denote observations.

### Machine Learning-Assisted Detection

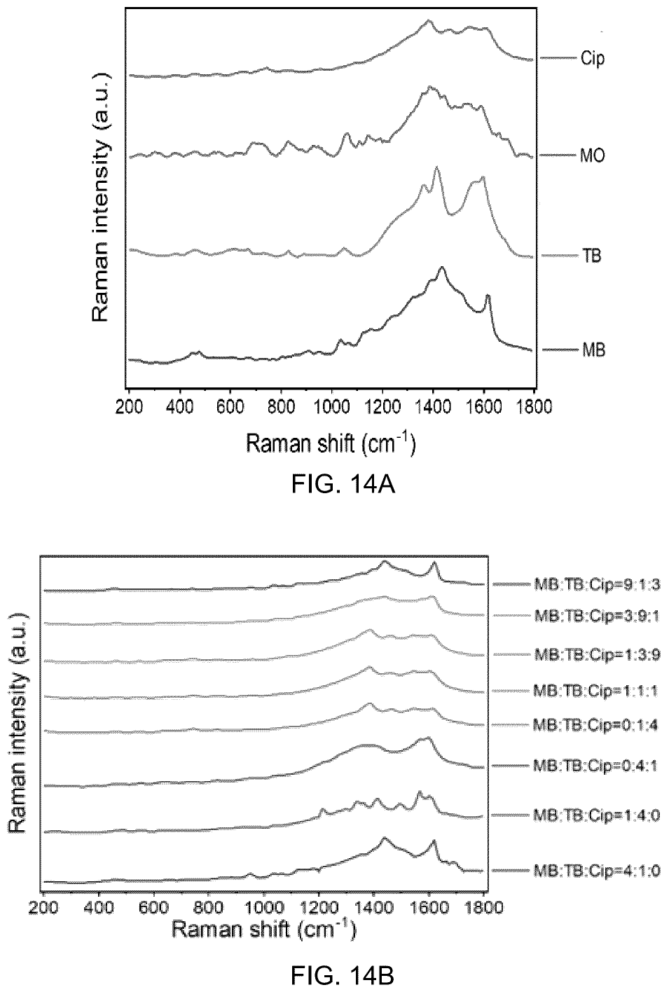

Raman spectra obtained by AgNP-ZnONR-SNF chip may contain intrinsic vibrational fingerprints that can aid in analyte identification, as shown in FIG. 14A. Specifically, FIG. 14A displays the SERS spectra of Cip, MO, TB, and MB. However, accurately characterizing and explaining the molecular structure of mixtures with overlapping characteristic peaks can be challenging, as shown in FIG. 14B. Specifically FIG. 14B displays the combinations of MB, TB, and Cip. To address this issue, a combination of deep neural network and databases was utilized in embodiments disclosed herein. The deep neural network consisted of a Laplacian operator and a four-layer fully connected NN structure that provided both qualitative and quantitative detection results. Although pre-processing is commonly used with Raman detection models, the inherent complexity of Raman spectra and the background noise often result in weak signals. To strengthen the useful information, the Laplacian operator was used. The marginal data points were eliminated, and the raw data of Raman spectrum underwent a Laplacian operator, which is a second-order central difference formula, to enhance the signal gradient, revealing the Raman peak information. The strengthened data was then fed into a four-layer fully connected NN structure with sigmoid/ReLU activation between each linear layer.

In this example, the four-layer fully connected NN included an input layer, two hidden layers, and an output layer, where each neuron was one layer is connected to every neuron in the next layer. This type of architecture allows information to flow through the network, with the hidden layers enabling the model to learn complex patterns from the input data. The activation functions between each two layers make the network capable of capturing more sophisticated relationships in the data. The size of each layer is 128, 64, 32, 16. The four-layer NN may be followed by a two-layer RNN. The NN structure and training parameters are shown above in Table 1.

### Neural Network (NN) Classification

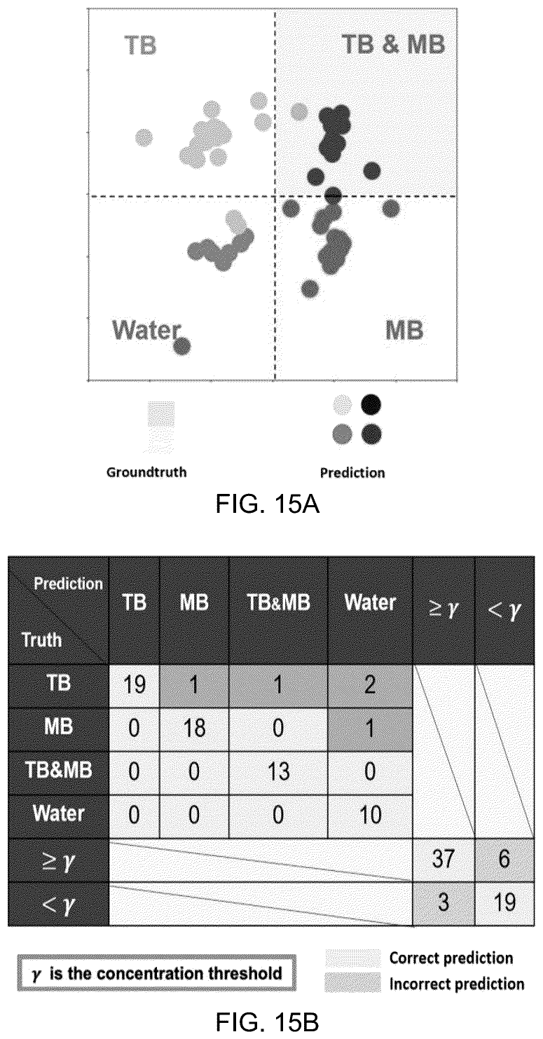

The classification was tested with Raman spectra of mixed dye of two types (MB, TB, MB and TB at different ratios, and water only) at multiple concentrations sensed from the AgNP-ZnONRSNF chip. In this example, the output of the NN was a five-digit tensor. The first four digits of the output tensor correspond to the dye type, indicating the possibility of the four classes that the sample component belongs to. The four included water, MB only, TB only, and the mixture of TB and MB. The last digit of the output tensor indicated whether the concentration of a specific analyte is above a typical cutoff threshold: [1] above the threshold and [0] indicates a smaller concentration. In this example, the concentration threshold was set as 5 μg/mL. The qualitative detection results are illustrated in FIG. 15A, where the positions of the points indicate the predicted sample components, and the color of the points indicates their true components. The statistical results are shown in FIG. 15B, with an accuracy of 92.3% in qualitative detection and 90.8% in quantitative detection. A comprehensive summary containing 100 tests is listed in Table 2.

TABLE 2
Prediction results from regressor model

| | TB | TB | MB | MB | Cip | Cip |
|---|---|---|---|---|---|---|
| Testing | prediction | truth | prediction | truth | prediction | truth |
| 1 | 0.00 | 0 | 1.00 | 1 | 9.00 | 9 |
| 2 | 0.07 | 0 | 3.61 | 4 | 0.91 | 1 |
| 3 | 1.00 | 1 | 1.00 | 1 | 1.00 | 1 |
| 4 | 0.40 | 0 | −0.06 | 0 | 1.33 | 1 |
| 5 | 0.01 | 0 | 1.00 | 1 | 9.04 | 9 |
| 6 | 5.11 | 5 | 4.91 | 5 | 0.00 | 0 |
| 7 | 0.00 | 0 | 0.99 | 1 | 9.02 | 9 |
| 8 | 1.01 | 1 | 1.00 | 1 | 1.00 | 1 |
| 9 | 1.49 | 1 | 0.10 | 0 | 1.11 | 1 |
| 10 | 2.03 | 2 | 7.96 | 8 | 0.01 | 0 |
| 11 | 1.62 | 2 | 0.35 | 0 | 1.44 | 1 |
| 12 | 1.89 | 3 | 0.49 | 1 | 5.27 | 9 |
| 13 | 0.09 | 0 | 1.05 | 1 | 4.14 | 4 |
| 14 | 1.00 | 1 | 9.01 | 9 | 3.01 | 3 |
| 15 | 4.25 | 9 | 2.20 | 3 | 1.02 | 1 |
| 16 | 1.50 | 1 | −0.08 | 0 | 1.06 | 1 |
| 17 | 1.01 | 1 | 9.02 | 9 | 3.01 | 3 |
| 18 | 0.00 | 0 | 1.00 | 1 | 9.00 | 9 |
| 19 | 1.01 | 1 | 0.00 | 0 | 1.00 | 1 |
| 20 | 5.11 | 5 | 4.91 | 5 | 0.00 | 0 |
| 21 | 10.00 | 10 | 0.00 | 0 | 0.00 | 0 |
| 22 | 2.27 | 3 | 7.76 | 7 | 0.01 | 0 |
| 23 | 0.08 | 0 | −0.11 | 0 | 0.73 | 0 |
| 24 | 2.58 | 2 | 7.39 | 8 | 0.00 | 0 |
| 25 | 0.08 | 1 | 9.86 | 9 | −0.01 | 0 |
| 26 | 1.02 | 1 | 8.98 | 9 | 0.00 | 0 |
| 27 | 1.88 | 3 | 1.26 | 1 | 7.19 | 9 |
| 28 | 8.02 | 8 | 1.97 | 2 | 0.00 | 0 |
| 29 | 2.50 | 3 | 2.44 | 1 | 2.89 | 9 |
| 30 | −0.23 | 0 | 0.48 | 1 | 7.93 | 9 |
| 31 | 1.66 | 2 | 0.73 | 0 | 1.65 | 1 |
| 32 | 1.62 | 1 | 0.04 | 0 | 1.41 | 1 |
| 33 | 2.05 | 2 | 8.03 | 7.5 | 0.03 | 0 |
| 34 | 1.39 | 2 | 0.04 | 0 | 0.90 | 1 |
| 35 | 4.11 | 4 | −0.02 | 0 | 1.02 | 1 |
| 36 | 2.02 | 2 | 0.99 | 1 | 4.01 | 4 |
| 37 | 0.01 | 0 | 1.00 | 1 | 4.01 | 4 |
| 38 | 2.93 | 3 | 7.06 | 7 | 0.00 | 0 |
| 39 | 2.97 | 3 | 7.03 | 7 | 0.00 | 0 |
| 40 | 1.81 | 1 | 8.25 | 9 | 0.02 | 0 |
| 41 | 1.84 | 2 | 8.22 | 8 | 0.02 | 0 |
| 42 | 5.11 | 5 | 4.91 | 5 | 0.00 | 0 |
| 43 | 1.01 | 1 | 8.96 | 9 | 0.00 | 0 |
| 44 | 0.91 | 1 | −0.07 | 0 | 1.12 | 4 |
| 45 | 1.03 | 1 | 9.00 | 9 | 3.00 | 3 |
| 46 | 5.11 | 5 | 4.91 | 5 | 0.00 | 0 |
| 47 | 1.01 | 1 | 0.00 | 0 | 2.01 | 2 |
| 48 | 1.01 | 1 | 3.99 | 4 | 2.01 | 2 |
| 49 | 0.00 | 0 | 0.00 | 0 | 0.68 | 0 |
| 50 | 3.13 | 3 | 6.87 | 7 | 0.00 | 0 |
| 51 | 7.04 | 7 | 2.97 | 3 | 0.00 | 0 |
| 52 | 1.01 | 1 | 3.99 | 4 | 2.01 | 2 |
| 53 | 0.01 | 0 | 1.01 | 1 | 9.00 | 9 |
| 54 | 1.28 | 2 | 1.68 | 0 | 1.81 | 1 |
| 55 | −0.08 | 0 | 0.07 | 0 | 1.07 | 1 |
| 56 | 9.08 | 9 | 2.87 | 3 | 0.07 | 1 |
| 57 | 3.06 | 3 | 6.97 | 7 | 0.00 | 0 |
| 58 | 7.02 | 7 | 3.00 | 3 | 0.00 | 0 |
| 59 | 2.42 | 2 | 7.54 | 7.5 | −0.01 | 0 |
| 60 | 0.01 | 0 | 1.01 | 1 | 8.89 | 9 |
| 61 | 2.02 | 2 | 1.00 | 1 | 4.03 | 4 |
| 62 | 1.01 | 1 | 9.00 | 9 | 3.01 | 3 |
| 63 | 9.12 | 9 | 2.49 | 3 | 0.80 | 1 |
| 64 | 7.15 | 7 | 2.80 | 2.5 | −0.01 | 0 |
| 65 | 4.89 | 9 | 2.30 | 3 | 0.87 | 1 |
| 66 | 6.19 | 6 | 3.80 | 4 | 0.01 | 0 |
| 67 | 3.98 | 4 | 0.00 | 0 | 0.99 | 1 |
| 68 | −0.08 | 0 | 10.02 | 10 | 0.00 | 0 |
| 69 | 1.04 | 1 | 0.46 | 0 | 1.20 | 2 |
| 70 | 1.01 | 1 | 0.00 | 0 | 1.00 | 1 |
| 71 | 3.02 | 3 | 6.97 | 7 | 0.00 | 0 |
| 72 | 2.01 | 2 | 1.00 | 1 | 4.00 | 4 |
| 73 | 7.03 | 7 | 2.96 | 3 | −0.01 | 0 |
| 74 | −0.05 | 0 | 2.31 | 1 | 1.14 | 9 |
| 75 | 0.00 | 0 | 4.05 | 4 | 1.04 | 1 |
| 76 | 9.39 | 9 | 2.25 | 3 | 0.85 | 1 |
| 77 | −0.03 | 0 | 3.81 | 4 | 1.00 | 1 |
| 78 | 8.02 | 8 | 1.99 | 2 | 0.01 | 0 |
| 79 | 2.10 | 0 | 8.00 | 10 | 0.02 | 0 |
| 80 | 6.98 | 7 | 3.02 | 3 | 0.00 | 0 |
| 81 | 9.02 | 9 | 1.00 | 1 | 0.00 | 0 |
| 82 | 1.48 | 2 | 1.18 | 1 | 2.23 | 4 |
| 83 | 3.01 | 3 | 6.97 | 7 | 0.00 | 0 |
| 84 | 1.66 | 1 | 0.33 | 1 | 0.98 | 1 |
| 85 | 5.11 | 5 | 4.91 | 5 | 0.00 | 0 |
| 86 | 9.69 | 10 | 0.29 | 0 | −0.02 | 0 |
| 87 | 9.89 | 10 | 0.12 | 0 | 0.00 | 0 |
| 88 | 1.01 | 1 | 0.00 | 0 | 1.00 | 1 |
| 89 | 0.91 | 1 | 0.25 | 0 | 2.29 | 4 |
| 90 | 7.15 | 7 | 2.86 | 3 | 0.00 | 0 |
| 91 | 1.02 | 1 | 4.13 | 4 | 2.26 | 2 |
| 92 | 0.00 | 0 | 0.00 | 0 | 0.07 | 0 |
| 93 | 0.00 | 0 | 0.00 | 0 | 0.68 | 0 |
| 94 | 9.97 | 10 | 0.05 | 0 | 0.00 | 0 |
| 95 | 4.10 | 4 | 5.88 | 6 | 0.00 | 0 |
| 96 | −0.01 | 0 | 0.01 | 0 | 1.01 | 1 |
| 97 | −0.67 | 0 | 0.57 | 1 | 7.82 | 9 |
| 98 | 7.80 | 7 | 2.19 | 2.5 | 0.00 | 0 |
| 99 | 6.48 | 4 | 2.82 | 2 | 0.09 | 1 |
| 100 | 3.80 | 4 | 1.96 | 2 | 0.99 | 1 |

### NN Regression

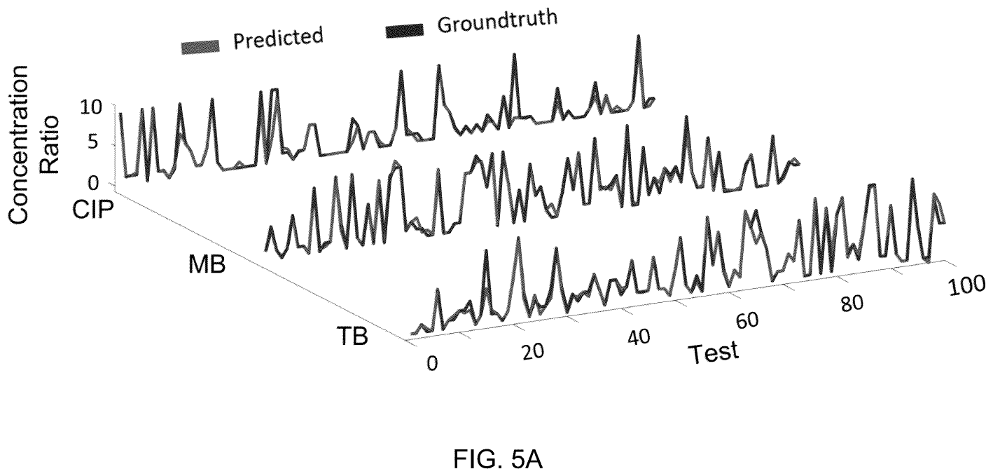

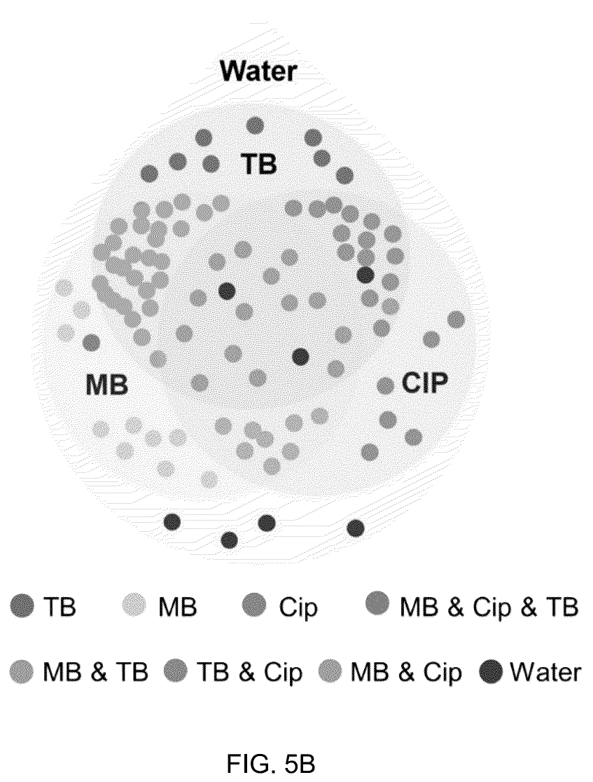

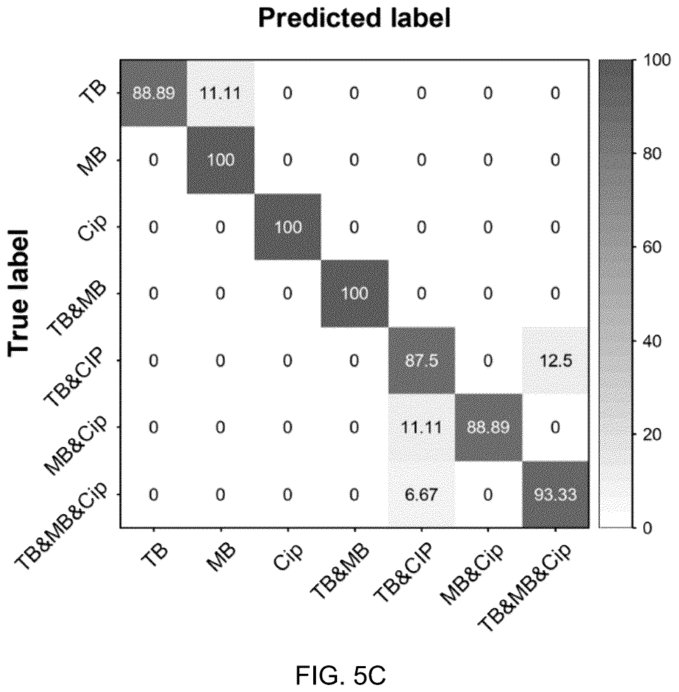

The regression model was further tested using Raman spectra obtained from mixtures of three components: two types of dyes (MB and TB) and one type of antibiotic (Cip) mixed at different concentration ratios. The concentration ratios of the detected analytes are displayed in FIG. 5A, which show an agreement with the ground truth curves with an average absolute error of 0.327 when the concentration ratio ranges from 0 to 10. The component of the sample could also be decided from the regression output, where the extremely low concentration ratio indicated that the specific analyte does not exist in the sample. For each analyte of the three, there was an existence (1) or non-existence (0) to create 8 different combinations in total. The qualitative detection of NN regression is shown in FIG. 5B, where the x-axis shows the TB component, the y-axis shows the MB component, and the z-axis shows the Cip component, creating 8 quadrants in total for different analyte component combinations. The results show a qualitative detection of 92% accuracy for analyte components. Compared with NN classification, the NN regression can directly output the concentration ratio, but had a slightly lower accuracy in qualitative detection, making these two models suitable for different scenarios. The statistical detection accuracy of each type of sample is shown in FIG. 5C, from mono-component samples to mixed opponent samples.

NN Classifier Combined with KNN

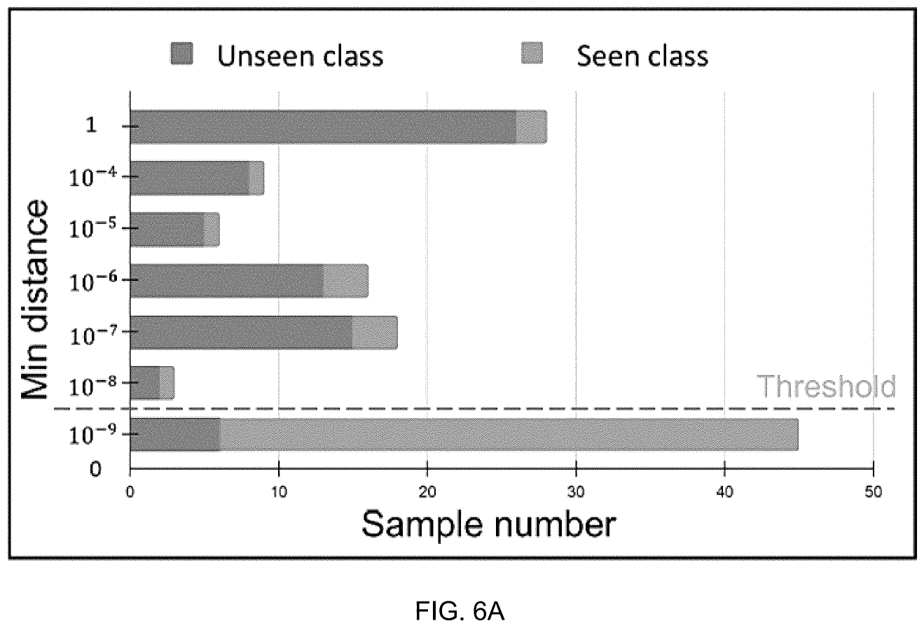

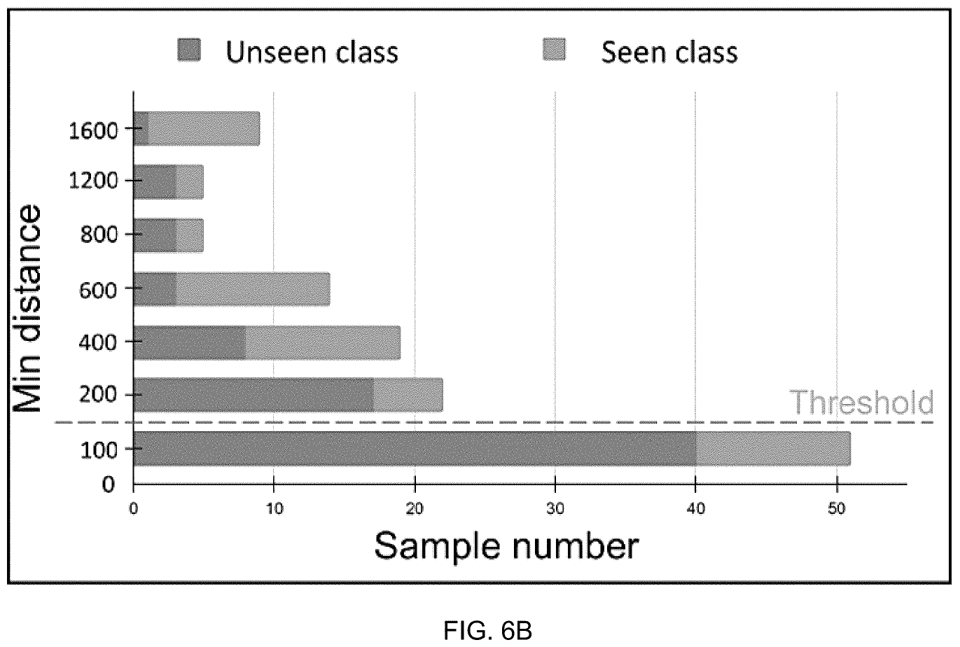

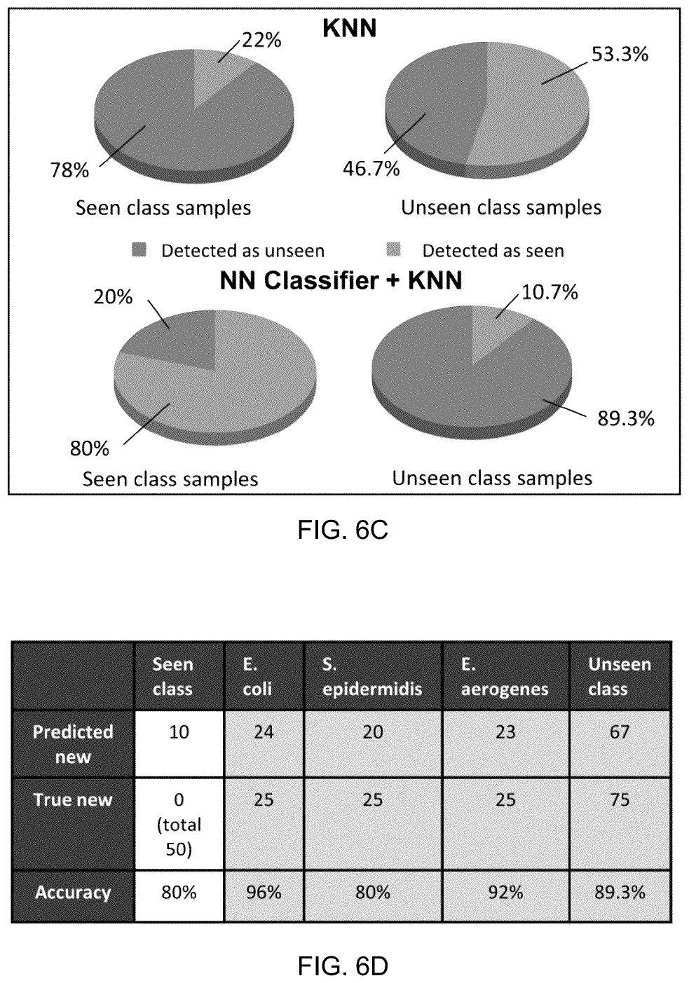

The KNN model was incorporated with the NN classifier for detecting novelty class points. The NN classifier and the KNN were trained with the same dataset for training the NN regressor, which was the mixtures of three components, MB, TB, and Cip, mixed at different concentration ratios, such as in this example. The output of the NN classifier was an 8-digit tensor to show the possibility of each class, and 8 clusters were established with the KNN clustering method through supervised training, as described herein. For testing cases, seen classes points (mixture of MB, TB, and CIP) as well as unseen classes points (3 bacteria including E. coli, S. epidermidis and E. aerogenes) were used. The minimum distance of each test point to the established clusters was calculated. Seen class points tended to have an extremely small distance to the cluster (in most cases the distance is 0), and the unseen class points were typically far from each established cluster. Therefore, a threshold could be employed to decide if the test point was an unseen class sample. The calculated minimum distances to the established clusters are shown in FIGS. 6A and 6B with thresholds to determine whether the test data points belong to seen classes or unseen classes. The results of the NN classifier combined with KNN (FIG. 6A) was compared to that of KNN alone (FIG. 6B). The results show that using the NN classifier and KNN, the majority of seen data points fall in the range with small distance (threshold) to the clusters, while very few of the unseen class points have that small distance. However, KNN alone does not provide the apparent dividing line for the two types of samples. The Detection results are shown in FIG. 6C, where NN classifier together with KNN reached 80% sensitivity (the true seen classes samples were correctly detected as seen) and 89.3% specificity (the true unseen classes samples were correctly detected as unseen), while the KNN method alone only had 22% sensitivity and 46.7% specificity. The detection results together with data distribution are displayed in FIG. 6D.

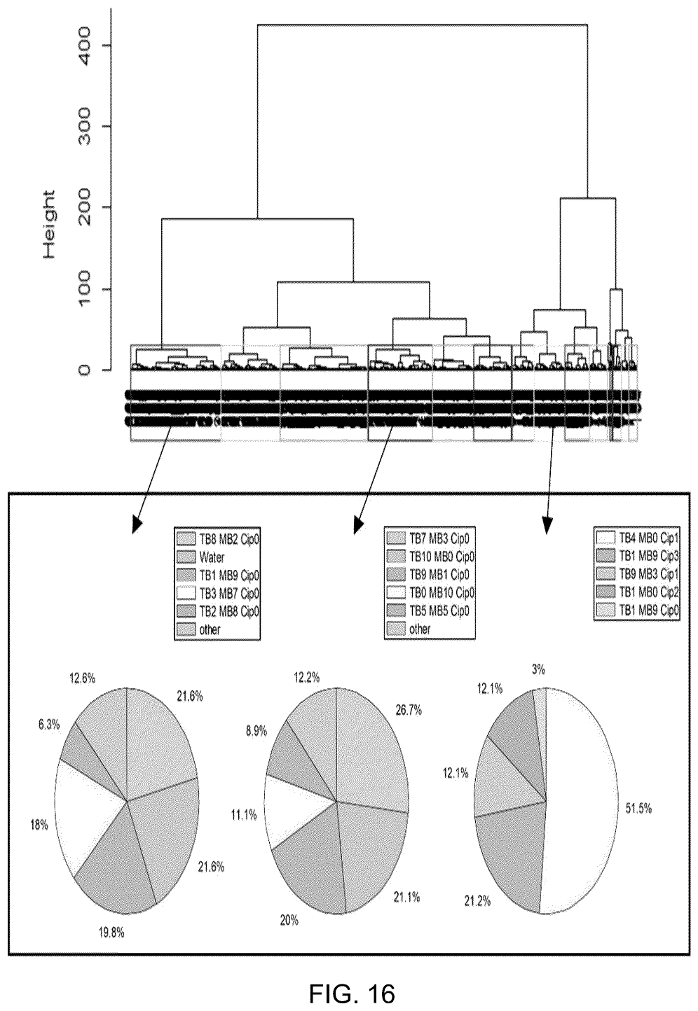

In comparison, standard analysis technique hierarchical cluster analysis (HCA) was used for classifying Cip, TB, and MB from 15 different mixtures. As shown in FIG. 16, many mixed pollutants from different mixtures were inaccurately grouped into one cluster. Specifically, FIG. 16 displays the hierarchical cluster analysis (HCA) of Raman spectra from 15 groups of mixtures containing Cip, TB, and MB. As shown, in HCA, the Euclidean distance and ward's algorithm were performed, and 15 clusters were selected. Further, FIG. 16 displays the proportion of groups from three randomly selected clusters. This suggests that SERS signal combined with DL is a reliable method to identify different organic pollutants in mixtures with high accuracy.

### Laplacian Operation

Detection of different analytes is critically based on the Raman peaks. The model disclosed herein implements the Laplacian operation to strengthen the useful information before the deep neural network without additional processing of Raman spectra. To validate the improvement from the Laplacian operator, the learning curves of NN regression were compared with and without Laplacian operation are shown in FIG. 16, which confirms that incorporating the Laplacian operation leads to a decrease in loss over the training.

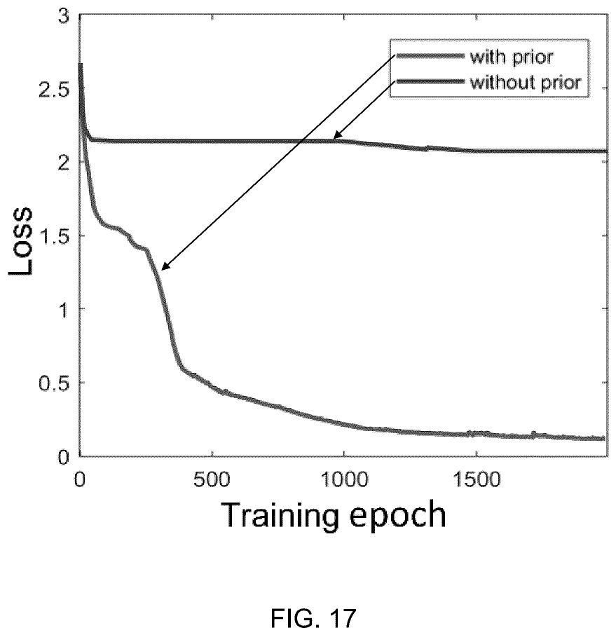

FIG. 17 displays the results of an ablation test to validate the rationality of central difference calculation prior treatment.

Although the present disclosure has been described with respect to one or more particular embodiments and/or examples, it will be understood that other embodiments and/or examples of the present disclosure may be made without departing from the scope of the present disclosure.

1. A method comprising detecting contaminants in a water sample using a machine learning algorithm, wherein the machine learning algorithm includes:
a Laplacian operator configured to extract Raman peak data;
a deep neural network; and
a K nearest neighbors (KNN) cluster model.

2. The method of claim 1, wherein the machine learning algorithm performs the detecting using the Raman peak data.

3. The method of claim 2, wherein the deep neural network includes a first output mode and a second output mode to output detection results.

4. The method of claim 3, wherein the first output mode is a classification mode to classify analyte components in the water sample and a concentration level of the water sample.

5. The method of claim 4, wherein the output is a tensor with a 0 or a 1, wherein the tensor indicates the concentration of the water sample.

6. The method of claim 5, wherein the output of the first mode is a five digit tensor, and the first four digits of the five digit tensor correspond to a dye type.

7. The method of claim 3, wherein the second output mode is a regression mode to detect a concentration of analytes present in the water sample.

8. The method of claim 7, wherein the second output is a tensor with a number, wherein the tensor indicates the concentration of the analytes present in the water sample.

9. The method of claim 4, wherein the KNN cluster model is combined with the classification mode to classify the analyte components in the water sample.

10. The method of claim 1, further comprising measuring the water sample using surface-enhanced Raman spectroscopy to generate the Raman peak data.

11. The method of claim 1, wherein the contaminant is an organic pollutant, an inorganic pollutant, a bacterial contaminant, or a virus.

12. A non-transitory computer readable medium storing a program configured to instruct a processor to execute the method of claim 1.

13. A method comprising:
providing a test system, wherein the test system includes:
a silicon nanofiber film;
a plurality of ZnO nanorods arranged in an array on the silicon nanofiber film;
a plurality of silver particles disposed on the plurality of ZnO nanorods;
applying a water sample onto the test system; and
detecting contaminants in the water sample using a machine learning algorithm, wherein the machine learning algorithm includes:
a Laplacian operator configured to extract Raman peak data;
a deep neural network; and
a K nearest neighbors (KNN) cluster model.

14. The method of claim 13, wherein the machine learning algorithm performs the detecting using the Raman peak data.

15. The method of claim 13, wherein the deep neural network includes a first output mode and a second output mode to output detection results.

16. The method of claim 15, wherein the first output mode is a classification mode to classify analyte components in the water sample and a concentration level of the water sample.

17. The method of claim 15, wherein the second output mode is a regression mode to detect a concentration of analytes present in the water sample.

18. The method of claim 13, wherein the KNN cluster model is combined with the classification mode to classify the analyte components in the water sample.

19. The method of claim 13, further comprising measuring the water sample on the test system using surface-enhanced Raman spectroscopy to generate the Raman peak data.

20. The method of claim 13, wherein the contaminant is an organic pollutant, an inorganic pollutant, a bacterial contaminant, or a virus.
